(19) United States
(12) Patent Application Publication
(10) Pub. No.: US 2025/0376453 A1
(43) Pub. Date: Dec. 11, 2025

(54) HYDROGENATION OF FURFURAL TO BIOFUEL USING A METAL NANOPARTICLE IMPREGNATED RED MUD CATALYST

(71) Applicant: KING FAHD UNIVERSITY OF PETROLEUM AND MINERALS, Dhahran (SA)

(72) Inventors: M. Nasiruzzaman SHAIKH, Dhahran (SA); Afnan Mohammed AJEEBI, Dhahran (SA); Md. Abdul AZIZ, Dhahran (SA)

(73) Assignee: KING FAHD UNIVERSITY OF PETROLEUM AND MINERALS, Dhahran (SA)

(21) Appl. No.: 18/735,294

(22) Filed: Jun. 6, 2024

(51) Int. Cl. C07D 307/44; B01J 8/02; B01J 23/89; B01J 35/45; B01J 35/61; B01J 37/04; B01J 37/08

(52) U.S. Cl. CPC C07D 307/44; B01J 8/0221; B01J 23/8906; B01J 35/45; B01J 35/612; B01J 35/613; B01J 37/04; B01J 37/088; B01J 2235/15

(57) ABSTRACT

A method of converting furfural to a conversion product includes introducing furfural, an alcohol solvent and a red mud-supported catalyst to a reactor and mixing to form a mixture. The method includes introducing a hydrogen-containing gas into the reactor and contacting with the mixture thereby reacting the hydrogen of the hydrogen-containing gas and furfural in the presence of the red mud-supported catalyst to form the conversion product. The red mud-supported catalyst is at least one of a red mud-supported rhodium (Rh@RM) catalyst, a red mud-supported iridium (Ir@RM) catalyst, and a red mud-supported ruthenium (Ru@RM) catalyst.

### STATEMENT OF ACKNOWLEDGEMENT

Support provided by the Interdisciplinary Research Center for Hydrogen and Energy Storage (IRC-HES), at King Fahd University of Petroleum and Minerals (KFUPM) for funding this work through Hydrogen Consortium (grant number H2FC2312) is gratefully acknowledged.

### BACKGROUND

### Technical Field

The present disclosure is directed to a red mud-supported catalyst and, more particularly, to a red mud supported rhodium (Rh@RM) catalyst; and directed to a method of converting furfural to a conversion product using the red mud-supported catalyst.

### Description of Related Art

The “background” description provided herein is to present the context of the disclosure generally. Work of the presently named inventors, to the extent it is described in this background section, as well as aspects of the description that may not otherwise qualify as prior art at the time of filing, are neither expressly nor impliedly admitted as prior art against the present invention.

Continuous fossil fuel consumption has had a tremendous environmental impact, necessitating an urgent transition to a renewable energy source derived from sustainable feedstock. Furthermore, biomass conversion reduces the reliance of society on hydrocarbon fossil fuels (such as oil, coal, and gas), particularly in the fuel and energy sector. Furfural is a crucial bio-based platform molecule containing an aldehyde group (—C═O) and a conjugated double-bond system. As a result, there is an opportunity for the development of various value-added chemicals. About 62% of furfural has been utilized industrially to produce furfuryl alcohol (FA) (See: A. Mandalika, L. Qin, T. K. Sato, T. Runge, Green Chem. 2014, 16, 2480-2489). Furfural hydrogenation activity and selectivity using Pd-based catalysts on acidic supports results in hydrogenation of FA to tetrahydrofurfuryl alcohol (THFA) (See: L. Liu, H. Lou, M. Chen, Appl Catal A Gen 2018, 550, 1-10). Additionally, FA and 2-methylfuran (MF) are produced during the hydrogenation of furfural in the presence of Pt-based catalysts, rather than FA or THFA (See: S. Bhogeswararao, D. Srinivas, J Catal 2015, 327, 65-77; C. Wang, J. Luo, V. Liao, J. D. Lee, T. M. Onn, C. B. Murray, R. J. Gorte, Catal Today 2018, 302, 73-79; R. V. Maligal-Ganesh, C. Xiao, T. W. Goh, L.-L. Wang, J. Gustafson, Y. Pei, Z. Qi, D. D. Johnson, S. Zhang, F. (Feng) Tao, W. Huang, ACS Catal 2016, 6, 1754-1763). Ru catalysts supported on carbon black, multi-walled carbon nanotubes, or activated carbon exhibited efficacy for furfural hydrogenation to FA (See: R. M. Mironenko, O. B. Belskaya, T. I. Gulyaeva, A. I. Nizovskii, A. V. Kalinkin, V. I. Bukhtiyarov, A. V. Lavrenov, V. A. Likholobov, Catal Today 2015, 249, 145-152]. A series of Ir-supported carbon (Ir/C) catalysts with 95% MF yield at low H₂ pressures (See: N. S. Date, A. M. Hengne, K.-W. Huang, R. C. Chikate, C. V. Rode, Green Chemistry 2018, 20, 2027-2037). However, research into to producing ethylfurfuryl ether (EFE) remains limited. This furanic ether has a low CO₂ emission footprint and a high-octane number, making it a fuel additive with blending properties and no adverse effects on engine performance. Typically, EFE is produced in two steps: first, hydrogenation of furfural (FF) to its corresponding alcohol (FA), followed by acid-catalyzed etherification of alcohol using ethyl alcohol as a solvent.

Although several catalysts have been developed for converting furfural to a conversion product, including EFE, these disclosed catalytic methods often involve a multi-step process and show poor selectivity towards EFE, leading to diminished yields or necessitating numerous purification steps. Therefore, there is a need to establish a catalytic process for converting furfural to EFE in a single step process with improved selectivity towards EFE.

Accordingly, it is one objective of the present disclosure to provide a method of converting furfural to a conversion product, including EFE, in the presence of a red mud supported catalyst. A second objective of the present disclosure is to provide a method of making the red mud supported catalyst.

### SUMMARY

In an exemplary embodiment, a method of converting furfural to a conversion product is described. The method includes introducing furfural, an alcohol solvent and a red mud-supported catalyst to a reactor and mixing to form a mixture. The method further includes introducing a hydrogen-containing gas into the reactor and contacting with the mixture thereby reacting the hydrogen of the hydrogen-containing gas and furfural in the presence of the red mud-supported catalyst to form the conversion product. In some embodiments, the red mud-supported catalyst is at least one of a red mud-supported rhodium (Rh@RM) catalyst, a red mud-supported iridium (Ir@RM) catalyst, and a red mud-supported ruthenium (Ru@RM) catalyst.

In some embodiments, the alcohol solvent is at least one selected from the group consisting of methanol, ethanol, propanol, butanol, pentanol, hexanol, heptanol, octanol, nonanol, decanol, undecanol, and dodecanol.

In some embodiments, the alcohol solvent is ethanol.

In some embodiments, the hydrogen-containing gas further includes an inert gas selected from the group consisting of nitrogen, argon, and helium.

In some embodiments, the method has a furfural conversion of at least 60% based on an initial weight of the furfural present in the mixture.

In some embodiments, the reactor is a fixed-bed reactor in the form of a cylindrical reactor including a top portion, a cylindrical body portion, a bottom portion, a housing having an open top and open bottom supportably maintained with the cylindrical body portion. In some embodiments, the red mud-supported catalyst is supportably retained within the housing permitting fluid flow therethrough. In some embodiments, at least one propeller agitator is disposed in the bottom portion of the reactor. In some embodiments, the bottom portion is cone shaped or pyramidal. In some embodiments, a plurality of recirculation tubes fluidly connects the bottom portion of the cylindrical reactor with the cylindrical body portion of the cylindrical reactor.

In some embodiments, the method includes reacting the hydrogen of the hydrogen-containing gas and furfural in the presence of the red mud-supported catalyst at a temperature of 80 to 160° C.

In some embodiments, the method includes reacting the hydrogen of the hydrogen-containing gas and furfural in the presence of the red mud-supported catalyst under a pressure ranging from 5 to 100 bar.

In some embodiments, the red mud-supported catalyst is a Rh@RM catalyst. The Rh@RM catalyst includes about 0.5 to 5 wt. % of Rh based on a total weight of the Rh@RM catalyst.

In some embodiments, the red mud-supported catalyst is a Rh@RM catalyst. In some embodiments, the Rh@RM catalyst includes irregular-shaped particles and needle-shaped particles having an average diameter of 30 to 80 nanometers (nm).

In some embodiments, the red mud-supported catalyst is a Rh@RM catalyst. In some embodiments, the Rh nanoparticles of the Rh@RM catalyst are uniformly distributed on surfaces of the Rh@RM catalyst. In some embodiments, the Rh nanoparticles have an average particle size of less than 1 nm.

In some embodiments, the conversion product includes furfuryl alcohol (FA), valeric acid (VA), tetrahydrofurfuryl alcohol (THFA), diethyl-furfuryl ether (Di-EFE), and ethyl furfurylether (EFE).

In some embodiments, the EFE is present in the conversion product in an amount of 30 to 80 wt. % based on the furfural conversion.

In an exemplary embodiment, a method of preparing the red mud-supported catalyst is described. The method includes calcining a red mud material at a temperature of 400 to 600° C. to form a calcined material; grinding and mixing a metal salt and the calcined material to form a precursor material; and heating the precursor material to form the red mud-supported catalyst.

In some embodiments, the red mud material is a waste product from an aluminum extraction process.

In some embodiments, the red mud material includes one or more crystalline phases selected from the group consisting of hematite, boehmite, anatase titania, and gibbsite, as determined by X-ray diffraction (XRD).

In some embodiments, the calcined material has a Brunauer-Emmett-Teller (BET) specific surface area of from 5 to 50 square meters per gram (m²/g).

In some embodiments, the metal salt is at least one selected from the group consisting of an iridium salt, a rhodium salt, and a ruthenium salt.

In some embodiments, the metal salt is present in the precursor material in an amount of 0.1 to 1 wt. % based on a total weight of the precursor material.

In some embodiments, the method includes heating the precursor material at a temperature of 350 to 450° C.

The foregoing general description of the illustrative present disclosure and the following detailed description thereof are merely exemplary aspects of the teachings of this disclosure and are not restrictive.

### BRIEF DESCRIPTION OF THE DRAWINGS

A more complete appreciation of this disclosure and many of the attendant advantages thereof may be readily obtained as the same becomes better understood by reference to the following detailed description when considered in connection with the accompanying drawings, wherein:

FIG. 1A is a flowchart illustrating a method of converting furfural to a conversion product, according to certain embodiments;

FIG. 1B is a flowchart illustrating a method of preparing a red mud (RM) supported catalyst (M@RM), according to certain embodiments;

FIG. 2 is a schematic illustration depicting a process of preparing the M@RM catalyst preparation, according to certain embodiments;

FIG. 3A is a field emission scanning electron microscopy (FESEM) image of RM-400 at 400° C., according to certain embodiments;

FIG. 3B is a FESEM image showing the presence of iron (Fe) and aluminium (Al) as a result of elemental mapping of RM-400, according to certain embodiments;

FIG. 3C is a FESEM image showing the presence of Fe as a result of elemental mapping of RM-400, according to certain embodiments;

FIG. 3D is a FESEM image showing the presence of Al as a result of elemental mapping of RM-400, according to certain embodiments;

FIG. 3E is a FESEM image of RM-500 at 500° C., according to certain embodiments;

FIG. 3F is a FESEM image showing the presence of Fe and Al as a result of elemental mapping of RM-500, according to certain embodiments;

FIG. 3G is a FESEM image showing the presence of Fe as a result of elemental mapping of RM-500, according to certain embodiments;

FIG. 3H is a FESEM image showing the presence of Al as a result of elemental mapping of RM-500, according to certain embodiments;

FIG. 3I is a FESEM image of RM-600 at 600° C., according to certain embodiments;

FIG. 3J is a FESEM image showing the presence of Fe and Al as a result of elemental mapping of RM-600, according to certain embodiments;

FIG. 3K is a FESEM image showing the presence of Fe as a result of elemental mapping of RM-600, according to certain embodiments;

FIG. 3L is a FESEM image showing the presence of Al as a result of elemental mapping of RM-600, according to certain embodiments;

FIG. 4A is a FESEM image of 0.5% Rh@RM-400 at 400° C., according to certain embodiments;

FIG. 4B is a FESEM image showing the presence of Fe, Al, and rhodium (Rh) as a result of elemental mapping of 0.5Rh@RM-400, according to certain embodiments;

FIG. 4C is a FESEM image showing the presence of Fe as a result of elemental mapping of 0.5%@RM-400, according to certain embodiments;

FIG. 4D is a FESEM image showing the presence of Al as a result of elemental mapping of 0.5%@RM-400, according to certain embodiments;

FIG. 4E is a FESEM image showing the presence of Rh as a result of elemental mapping of 0.5%@RM-400, according to certain embodiments;

FIG. 4F is a FESEM image of 0.5% Ir@RM-400 at 400° C., according to certain embodiments;

FIG. 4G is a FESEM image showing the presence of Fe, Al, and iridium (Ir) as a result of elemental mapping of 0.5% Ir@RM-400, according to certain embodiments;

FIG. 4H is a FESEM image showing the presence of Fe as a result of elemental mapping of 0.5% Ir@RM-400, according to certain embodiments;

FIG. 4I is a FESEM image showing the presence of Al as a result of elemental mapping of 0.5% Ir@RM-400, according to certain embodiments;

FIG. 4J is a FESEM image showing the presence of Ir as a result of elemental mapping of 0.5% Ir@RM-400, according to certain embodiments;

FIG. 4K is a FESEM image of 0.5% Ru@RM-400 at 400° C., according to certain embodiments;

FIG. 4L is a FESEM image showing the presence of Fe, Al, and ruthenium (Ru) as a result of elemental mapping of 0.5% Ru@RM-400, according to certain embodiments;

FIG. 4M is a FESEM image showing the presence of Fe as a result of elemental mapping of 0.5% Ru@RM-400, according to certain embodiments;

FIG. 4N is a FESEM image showing the presence of Al as a result of elemental mapping of 0.5% Ru@RM-400, according to certain embodiments;

FIG. 4O is a FESEM image showing the presence of Ru as a result of elemental mapping of 0.5% Ru@RM-400, according to certain embodiments;

FIG. 5A is a graph showing energy dispersive X-ray (EDX) analysis of RM-400, according to certain embodiments;

FIG. 5B shows EDX analysis of RM-500, according to certain embodiments;

FIG. 5C shows EDX analysis of RM-600, according to certain embodiments;

FIG. 5D shows EDX analysis of Rh@RM, according to certain embodiments;

FIG. 5E shows EDX analysis of Ir@RM, according to certain embodiments;

FIG. 5F shows EDX analysis of Ru@RM, according to certain embodiments;

FIG. 6A is a transmission electron microscopy (TEM) image of RM-400 at 200 nanometres (nm) magnification, according to certain embodiments;

FIG. 6B is a TEM image of RM-400 at 50 nm magnification, according to certain embodiments;

FIG. 6C is a high-resolution transmission electron microscopy (HRTEM) image of RM-400, according to certain embodiments;

FIG. 6D is a selected area electron diffraction (SAED) image of RM-400, according to certain embodiments;

FIG. 6E is a TEM image of Rh@RM-400 at 200 nm magnification, according to certain embodiments;

FIG. 6F is a TEM image of Rh@RM-400 at 50 nm magnification, according to certain embodiments;

FIG. 6G is a HRTEM image of Rh@RM-400, according to certain embodiments;

FIG. 6H is a SAED image of Rh@RM-400, according to certain embodiments;

FIG. 6I is a TEM image of Ir@RM-400 at 200 nm magnification, according to certain embodiments;

FIG. 6J is a TEM image of Ir@RM-400 at 50 nm magnification, according to certain embodiments;

FIG. 6K is a HRTEM image of Ir@RM-400, according to certain embodiments;

FIG. 6L is a SAED image of Ir@RM-400, according to certain embodiments;

FIG. 6M is a TEM image of Ru@RM-400 at 200 nm magnification, according to certain embodiments;

FIG. 6N is a TEM image of Ru@RM-400 at 50 nm magnification, according to certain embodiments;

FIG. 6O is a HRTEM image of Ru@RM-400, according to certain embodiments;

FIG. 6P is a SAED image of Ru@RM-400, according to certain embodiments;

FIG. 7A shows X-ray diffraction (XRD) analysis of RM-400, RM-500, and RM-600, according to certain embodiments;

FIG. 7B shows XRD analysis of 0.5% M@RM-400, according to certain embodiments;

FIG. 7C shows XRD analysis of x % Rh@RM-400, according to certain embodiments;

FIG. 8A shows Fourier transform infrared (FTIR) spectrum for RM-400, RM-500, and RM-600, according to certain embodiments;

FIG. 8B shows FTIR spectrum for RM-400 and 1% Rh@RM-400, according to certain embodiments;

FIG. 8C shows inductively coupled plasma (ICP) spectroscopy analysis of natural RM, RM-400, RM-500, and RM-600, according to certain embodiments;

FIG. 9A shows temperature programmed reducibility of RM-400, RM-500, RM-600, and 1% Rh@RM-400 using H₂, according to certain embodiments;

FIG. 9B shows N₂-adsorption isotherm of pure RM, RM-400, RM-500, and RM-600, according to certain embodiments;

FIG. 9C shows adsorption isotherm of 1% Rh@RM-400, according to certain embodiments;

FIG. 10A shows X-ray photoelectron spectroscopy (XPS) analysis of Fe for RM-400, according to certain embodiments;

FIG. 10B shows XPS analysis of Fe for RM-500, according to certain embodiments;

FIG. 10C shows XPS analysis of Fe for RM-600, according to certain embodiments;

FIG. 10D shows XPS analysis of Fe for 1% Rh@RM-400, according to certain embodiments;

FIG. 10E shows XPS analysis of Rh, according to certain embodiments;

FIG. 10F depicts possible routes for obtaining various oxidation products and hydrogenation products from biomass-derived furfural as a platform molecule, according to certain embodiments;

FIG. 11A shows effect of calcination temperature on a furfural hydrogenation reaction at 120° C. for 24 hours (h) using ethanol as a solvent, according to certain embodiments;

FIG. 11B shows effect of H₂ pressure on the furfural hydrogenation reaction at 120° C. for 24 h using ethanol as solvent, according to certain embodiments;

FIG. 12A shows catalytic evaluation of RM-400 catalyst at a temperature of under 140° C. using 1 millimole (mmol) of furfural in ethanol as a solvent for a duration of 24 h, according to certain embodiments;

FIG. 12B shows catalytic evaluation of RM-400 catalyst at a pressure under 50 bar using 1 mmol of furfural in ethanol as a solvent for a duration of 24 h, according to certain embodiments;

FIG. 13A shows the effect of loading 0.5 weight percentage (wt. %) of Ir, Ru, and Rh on RM-400 at 120° C. under 50 bar H₂, according to certain embodiments;

FIG. 13B shows the effect of a varied amount of Rh loading on RM-400 at 120° C. under 50 bar H₂, according to certain embodiments;

FIG. 13C shows the effect of H₂ pressure on conversion and selectivity using 1% Rh@RM-400 catalyst, according to certain embodiments;

FIG. 13D shows the effect of temperature on conversion and selectivity for the furfural hydrogenation reaction with 1% Rh@RM-400 catalyst, according to certain embodiments;

FIG. 14A depicts gas chromatography (GC) results of hydrogenation of the furfural at 120° C., under 50 bar H₂ pressure in dichloromethane anhydrous (DCM) as a solvent in 24 h using RM as a catalyst, according to certain embodiments;

FIG. 14B depicts the product of FIG. 14A (peak at Rₜ=3.2), as identified by gas chromatography/mass spectrometry (GCMS), according to certain embodiments.

FIG. 14C depicts GC results of hydrogenation of the furfural at 120° C., under 50 bar H₂ pressure in ethanol as a solvent in 24 h using RM as a catalyst, according to certain embodiments;

FIG. 14D depicts the product of FIG. 14C (peak at Rₜ=4.38), identified by GCMS, according to certain embodiments;

FIG. 14E depicts GC results of hydrogenation of the furfural at 120° C., under 50 bar H₂ pressure in ethanol as a solvent in 24 h using 0.5% Ir@RM as a catalyst, according to certain embodiments;

FIG. 14F depicts the product of FIG. 14E (peak at Rₜ=4.42), identified by GCMS, according to certain embodiments;

FIG. 14G depicts GC results of hydrogenation of the furfural at 120° C., under 50 bar H₂ pressure in ethanol as a solvent in 24 h using 0.5% Rh@RM as a catalyst, according to certain embodiments;

FIG. 14H depicts the product of FIG. 14G, identified by GCMS (peak at Rₜ=4.42), according to certain embodiments;

FIG. 14I depicts GC results of hydrogenation of the furfural at 120° C., under 50 bar H₂ pressure in ethanol as a solvent in 24 h using 0.5% Ru@RM as a catalyst, according to certain embodiments;

FIG. 14J depicts the product of FIG. 14I (peak at Rₜ=4.399), identified by GCMS, according to certain embodiments;

FIG. 14K depicts GC results of hydrogenation of the furfural at 120° C., under 50 bar H₂ pressure in ethanol as a solvent in 24 h using 1% Rh@RM as a catalyst, according to certain embodiments;

FIG. 14L depicts the product of FIG. 14K (peak at Rₜ=4.407), identified by GCMS, according to certain embodiments;

FIG. 14M depicts GC results of hydrogenation of the furfural at 120° C., under 50 bar H₂ pressure in ethanol as a solvent in 24 h using 3% Rh@RM as a catalyst, according to certain embodiments;

FIG. 14N depicts the product of FIG. 14M (peak at Rₜ=4.42), identified by GCMS, according to certain embodiments;

FIG. 140 depicts GC results of hydrogenation of the furfural at 120° C., under 50 bar H₂ pressure in ethanol as a solvent in 24 h using 5% Rh@RM as a catalyst, according to certain embodiments;

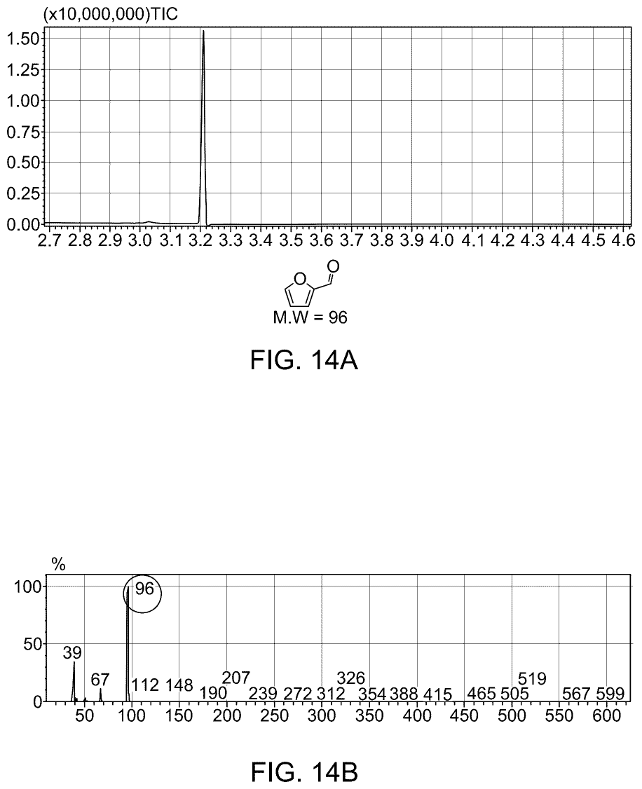

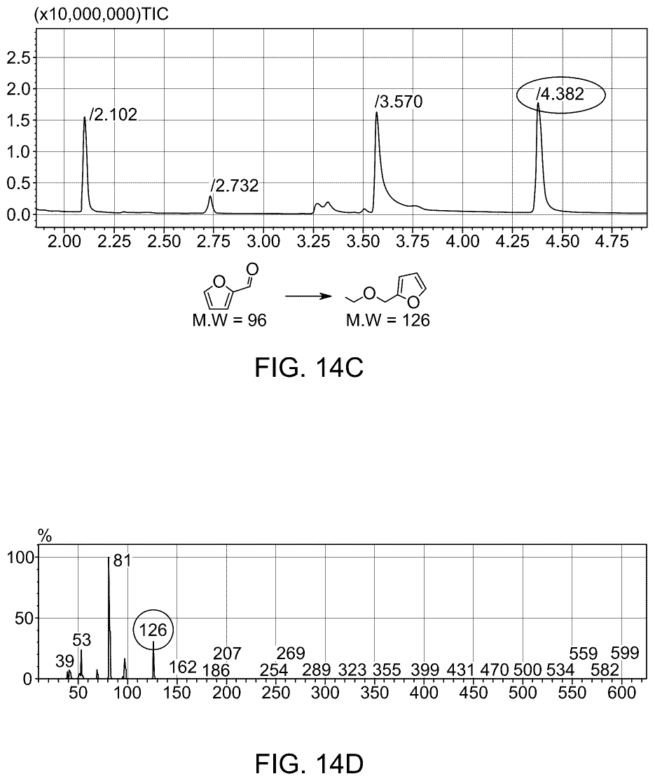

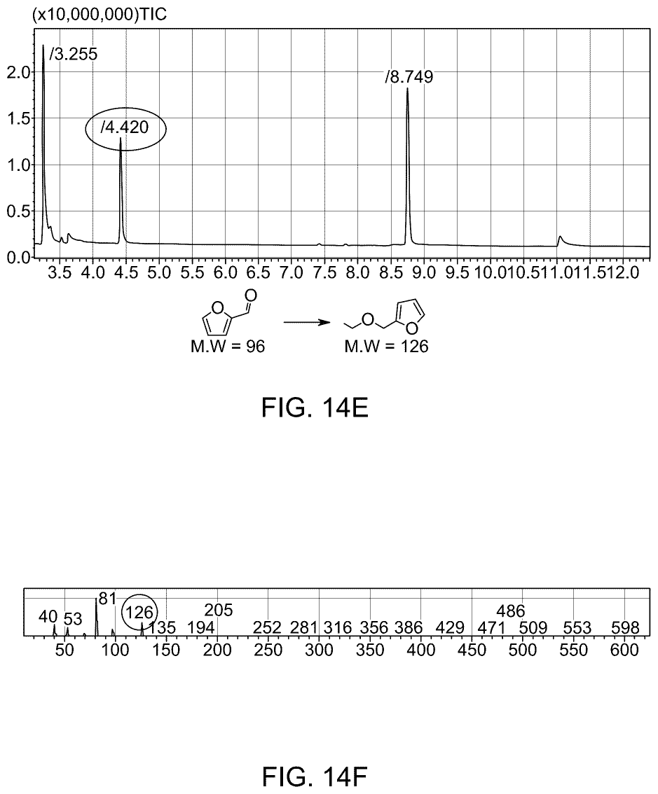

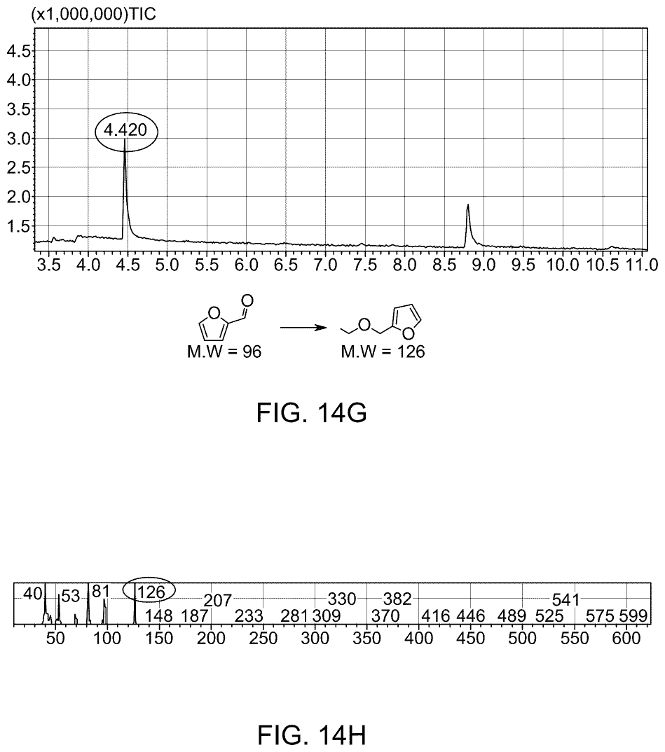

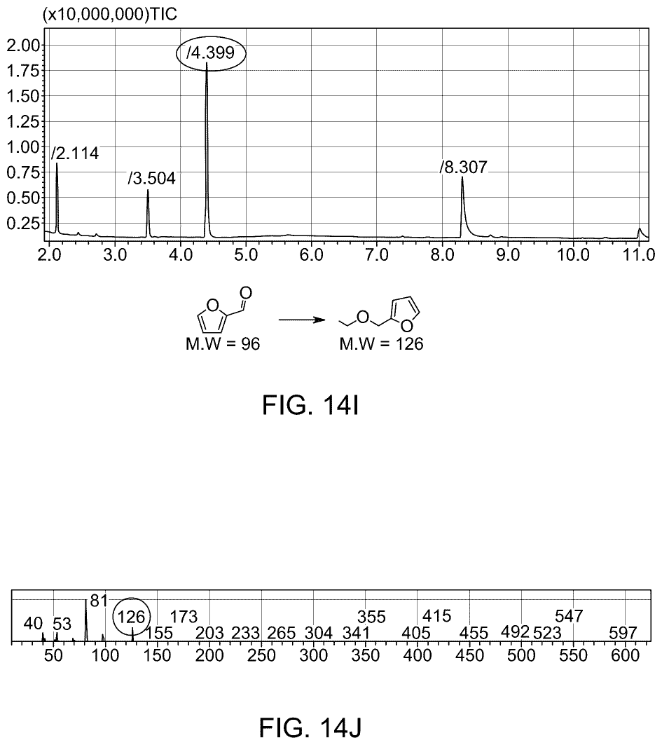

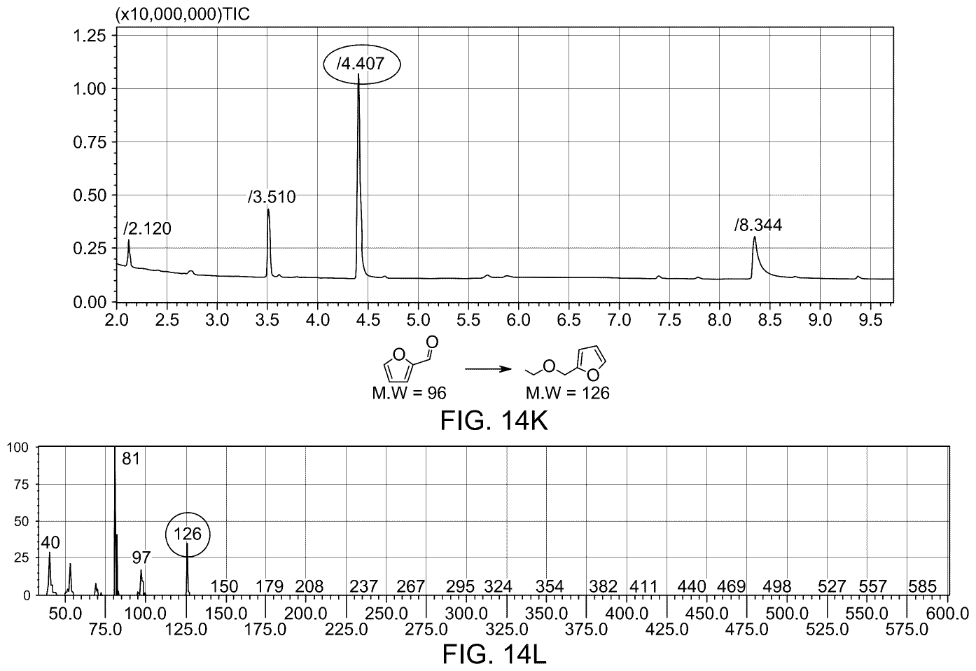

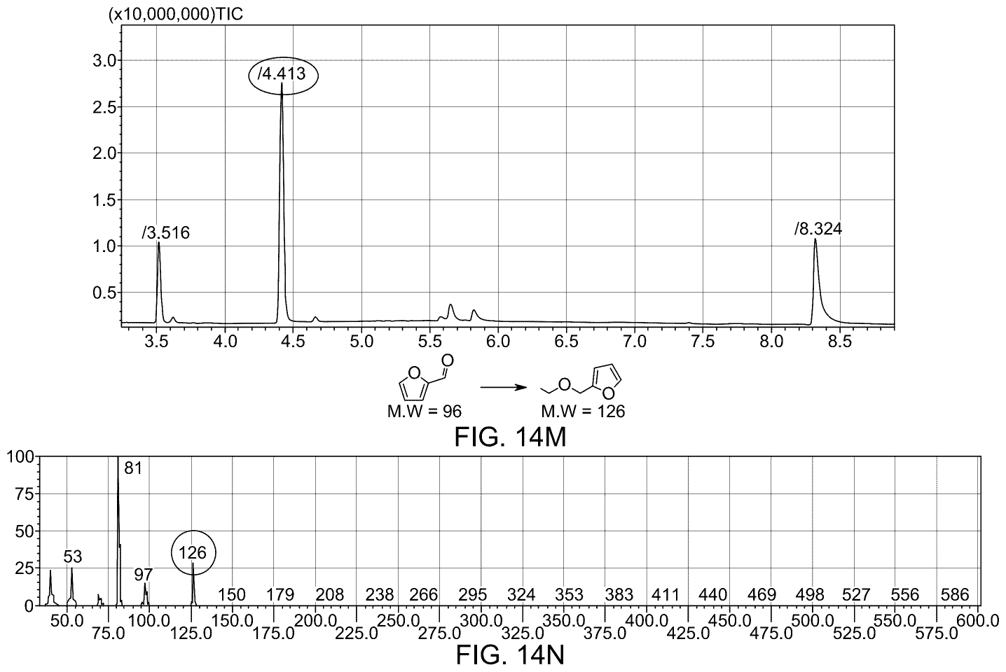

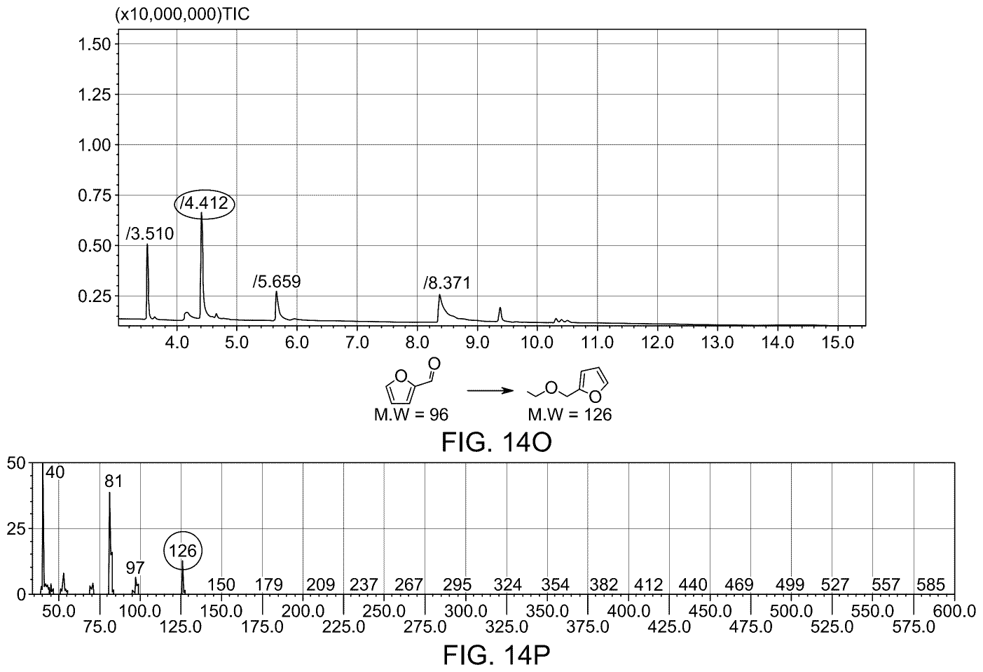

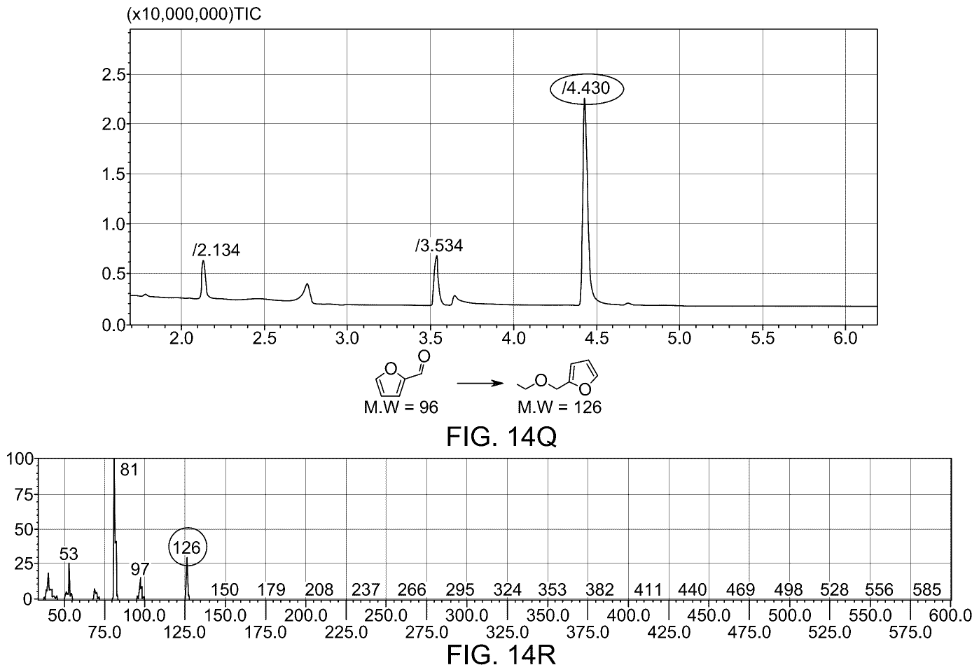

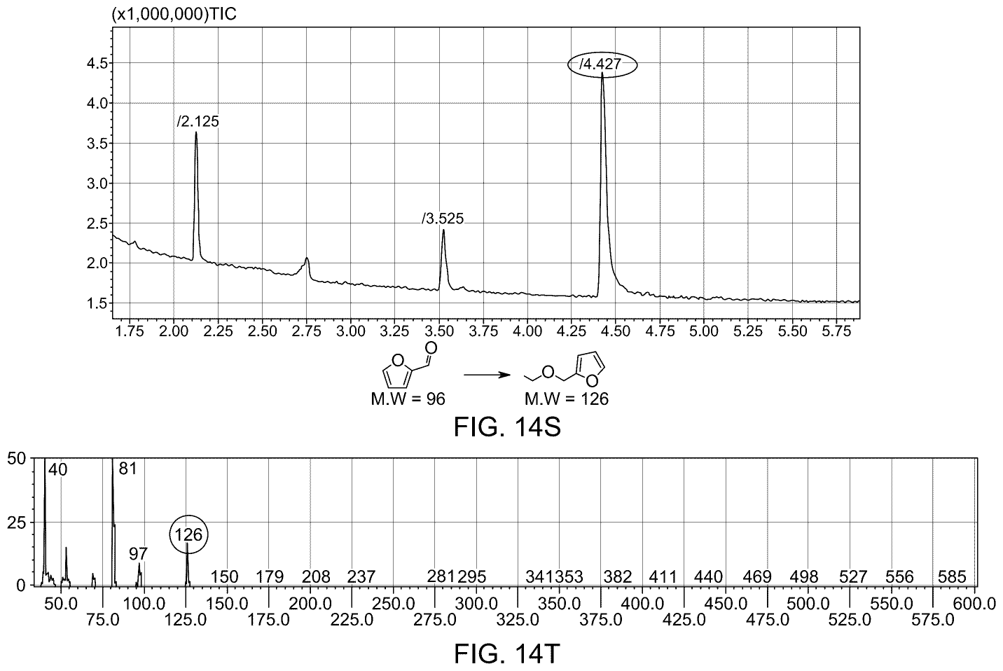

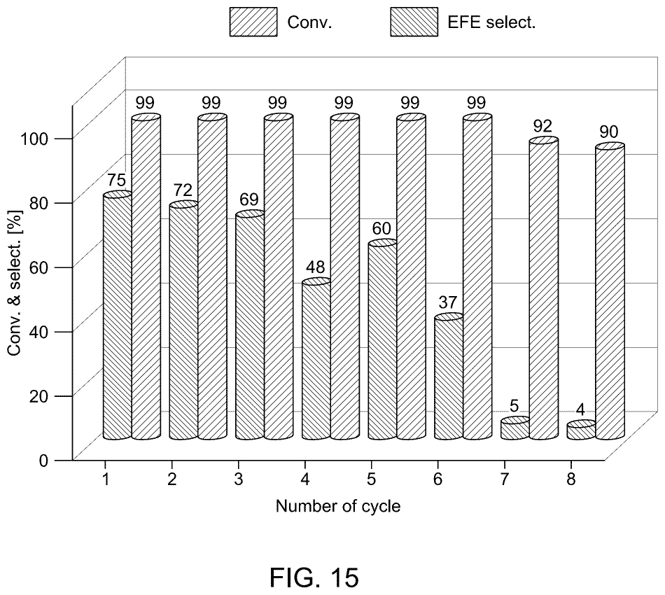

FIG. 14P depicts the product of FIG. 14O (peak at Rₜ=4.412), identified by GCMS, according to certain embodiments;

FIG. 14Q depicts GC results of hydrogenation of the furfural at 120° C., under 30 bar H₂ pressure in ethanol as a solvent in 24 h using 1% Rh@RM as a catalyst, according to certain embodiments;

FIG. 14R depicts the product of FIG. 14Q (peak at Rₜ=4.430), identified by GCMS, according to certain embodiments;

FIG. 14S depicts GC results of hydrogenation of the furfural at 120° C., under 20 bar H₂ pressure in ethanol as a solvent in 24 h using 1% Rh@RM as a catalyst, according to certain embodiments;

FIG. 14T depicts the product of FIG. 14S (peak at Rₜ=4.427), identified by GCMS, according to certain embodiments; and

FIG. 15 is a plot showing reusability of the 1% Rh@RM-400 catalyst in furfural hydrogenation at 120° C. under 30 bar H₂ pressure, according to certain embodiments.

### DETAILED DESCRIPTION

When describing the present disclosure, the terms used are to be construed in accordance with the following definitions, unless a context dictates otherwise. Embodiments of the present invention will now be described more fully hereinafter with reference to the accompanying drawings wherever applicable, in that some, but not all embodiments of the disclosure are shown.

In the drawings, reference numerals designate identical or corresponding parts throughout the several views. Further, as used herein, the words “a,” “an” and the like generally carry a meaning of “one or more,” unless stated otherwise.

As used herein, the words “about,” “approximately,” or “substantially similar” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), +/−15% of the stated value (or range of values), or +/−20% of the stated value (or range of values). Within the description of this disclosure, where a numerical limit or range is stated, the endpoints are included unless stated otherwise. Also, all values and subranges within a numerical limit or range are specifically included as if explicitly written out.

The use of the terms “include,” “includes”, “including,” “have,” “has,” or “having” should be generally understood as open-ended and non-limiting unless specifically stated otherwise.

As used herein, the term “room temperature” or “ambient temperature” generally refers to a temperature in a range of 25 degrees Celsius (° C.)±3° C. in the present disclosure.

Aspects of the present disclosure are directed towards a method of converting bio-derived molecules, such as furfural (FF), into a conversion product (including but not limited to, ethyl furfuryl ethers) using a red mud (RM) material as a catalyst. The method produces ethyl furfuryl ethers from FF in a single step with improved selectivity, thereby circumventing the drawbacks of the art.

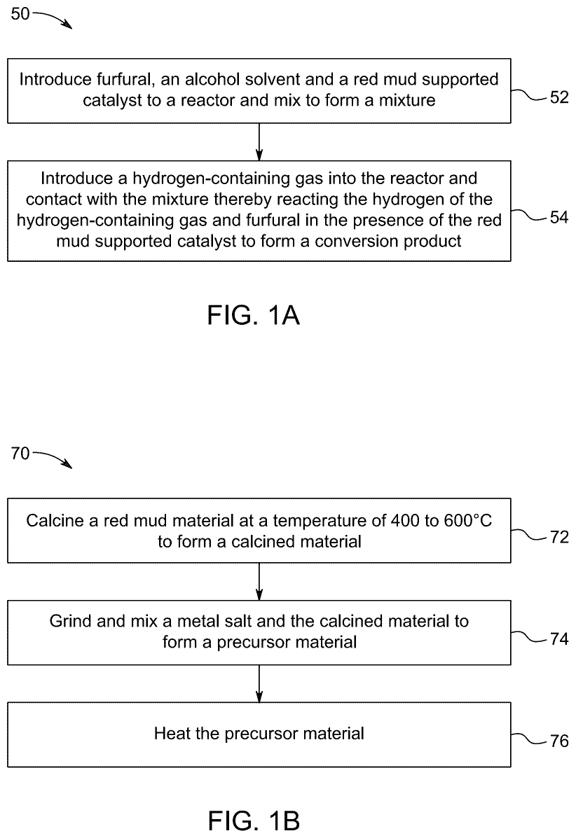

FIG. 1A illustrates a flow chart of a method 50 of converting furfural to a conversion product. The order in which the method 50 is described is not intended to be construed as a limitation, and any number of the described method steps can be combined in any order to implement the method 50. Additionally, individual steps may be removed or skipped from the method 50 without departing from the spirit and scope of the present disclosure.

At step 52, method 50 includes introducing furfural, an alcohol solvent, and a red mud-supported catalyst to a reactor and mixing to form a mixture. In some embodiments, the red mud-supported catalyst may include red mud material that is impregnated with at least one metal selected from rhodium to form red mud-supported rhodium (Rh@RM) catalyst; or iridium to form red mud-supported iridium (Ir@RM) catalyst; or ruthenium to form red mud-supported ruthenium (Ru@RM) catalyst. In a preferred embodiment, the red mud-supported catalyst is the Rh@RM catalyst. In some embodiments, the Rh@RM catalyst includes about 0.5 to 5 wt. %, more preferably 0.5%, 1%, 3%, and 5%, of rhodium (Rh) based on the total weight of the Rh@RM catalyst. Other ranges are also possible. In some embodiments, the Rh@RM catalyst includes irregular-shaped particles and needle-shaped rhodium nanoparticles having an average diameter of 30 to 80 nanometers (nm), preferably 40 to 70 nm, preferably 50 to 60 nm, or even more preferably about 55 nm. Other ranges are also possible. The Rh nanoparticles are uniformly distributed on the surfaces of the Rh@RM catalyst. The rhodium particles may be deposited wholly or partially over the red mud material in a uniform and continuous manner to form the Rh@RM catalyst. In some embodiments, the Rh nanoparticles have an average particle size of less than 10 nm, preferably less than 5 nm, preferably less than 3 nm, preferably less than 1 nm, or even more preferably less than 0.5 nm. Other ranges are also possible.

In some embodiments, the alcohol solvent is at least one selected from the group consisting of methanol, ethanol, propanol, butanol, pentanol, hexanol, heptanol, octanol, nonanol, decanol, undecanol, and dodecanol. In a preferred embodiment, the alcohol solvent is ethanol.

In some embodiments, the reactor is a fixed-bed reactor in the form of a cylindrical reactor including a top portion, a cylindrical body portion, a bottom portion, a housing having an open top and open bottom supportably maintained with the cylindrical body portion. In some embodiments, the red mud-supported catalyst is supportably retained within the housing permitting fluid flow therethrough. In some embodiments, at least one propeller agitator is disposed in the bottom portion of the reactor. In some embodiments, the bottom portion is cone shaped or pyramidal. In some embodiments, a plurality of recirculation tubes fluidly connects the bottom portion of the cylindrical reactor with the cylindrical body portion of the cylindrical reactor.

At step 54, the method 50 includes introducing a hydrogen-containing gas into the reactor and contacting with the mixture thereby reacting the hydrogen of the hydrogen-containing gas and furfural in the presence of the red mud-supported catalyst to form the conversion product. In some embodiments, the hydrogen-containing gas predominantly includes hydrogen. It may optionally further include an inert gas selected from nitrogen, argon, and helium. In some embodiments, the inert gas may be used in the following combinations: Ar/He, Ar/He/N₂, and N₂/He. The reaction between hydrogen of the hydrogen-containing gas and furfural in the presence of the red mud-supported catalyst is performed at a temperature of 80 to 160° C., more preferably 110 to 130° C., and more preferably 120° C., under a pressure ranging from 5 to 100 bar, more preferably 40 to 60 bar, and yet more preferably 50 bar, to yield the conversion product. Other ranges are also possible. In some embodiments, the conversion product can be one or more of furfuryl alcohol (FA), valeric acid (VA), tetrahydrofurfuryl alcohol (THFA), diethyl-furfuryl ether (Di-EFE), and ethyl furfurylether (EFE). In some embodiments, the furfural conversion by the method of present disclosure is at least 60% based on the initial weight of the furfural present in the mixture. In some embodiments, the EFE is present in the conversion product in an amount of 30 to 80 wt. %, more preferably 55 to 76 wt. %, more preferably 60% and 75% based on the furfural conversion. Other ranges are also possible.

FIG. 1B illustrates a flow chart of a method 70 of preparing the red mud-supported catalyst of the method 50. The order in which the method 70 is described is not intended to be construed as a limitation, and any number of the described method steps can be combined in any order to implement the method 70. Additionally, individual steps may be removed or skipped from the method 70 without departing from the spirit and scope of the present disclosure.

At step 72, the method 70 includes preparing the red mud-supported catalyst by calcining a red mud material at a temperature of 400 to 600° C., more preferably 400° C., more preferably 500° C., more preferably 600° C. to form a calcined material. As used herein, “red mud material” refers to an industrial waste product generated during the production of alumina. For example, such a waste product can comprise silica, aluminum, iron, calcium, and optionally titanium. It can also comprise an array of minor constituents such as Na, K, Cr, V, Ni, Co, Ba, Cu, Mn, Mg, Pb, and/or Zn etc. For example, the red mud material can comprise about 15 to about 80% by weight of Fe₂O₃, about 1 to about 35% by weight Al₂O₃, about 1 to about 65% by weight of SiO₂, about 1 to about 20% by weight of Na₂O, about 1 to about 20% by weight of CaO, and from 0 to about 35% by weight of TiO₂. Other ranges are also possible. According to another example, the red mud material can comprise about 30 to about 65% by weight of Fe₂O₃, about 10 to about 20% by weight Al₂O₃, about 3 to about 50° A) by weight of SiO₂, about 2 to about 10% by weight of Na₂O, about 2 to about 8% by weight of CaO, and from 0 to about 25% by weight of TiO₂. Other ranges are also possible. Typically, the red mud material can exist in many crystalline phases, such as hematite, boehmite, anatase titania, gibbsite, magnetite, siderite, and bauxite, as determined by X-ray diffraction (XRD). The person skilled in the art will understand that the composition of the red mud material can vary depending on the bauxite origin. The red mud material is calcined by heating it to a high temperature, under a restricted supply of ambient oxygen. This is performed to remove impurities or volatile substances and to incur thermal decomposition. In some embodiments, the calcination is carried out in a furnace, preferably equipped with a temperature control system, with a temperature range of 400 to 600° C., preferably 450 to 550° C., or even more preferably about 500° C. for 2 to 10 hours, preferably 3 to 8 hours, preferably 4 to 6 hours, or even more preferably about 5 hours. Other ranges are also possible.

At step 74, the method 70 includes grinding and mixing a metal salt and the calcined material to form a precursor material. The grinding may be carried out using any suitable means, for example, ball milling, blending, etc., using manual method (e.g., mortar) or machine-assisted methods such as using a mechanical blender, or any other apparatus known to those of ordinary skill in the art. In some embodiments, modes of mixing known to those of ordinary skill in the art, may include stirring, swirling, or a combination thereof. In some embodiments, the metal salt is at least one selected from the group consisting of an iridium salt, a rhodium salt, and a ruthenium salt. In some embodiments, the rhodium salt can be, for example, rhodium acetylacetonate, rhodium chloride, or rhodium sulfate. The iridium salt can be iridium chloride or iridium bromide. The ruthenium salt can be ruthenium(III) nitrosyl nitrate, ruthenium chloride, and ruthenium iodide. In some embodiments, the metal salt is present in the precursor material in an amount of 0.1 to 1 wt. %, preferably 0.2 to 0.8 wt. %, preferably 0.3 to 0.6 wt. %, or even more preferably about 0.5 wt. %, based on the total weight of the precursor material. Other ranges are also possible. In some embodiments, the calcined material has a Brunauer-Emmett-Teller (BET) specific surface area of from 5 to 50 square meters per gram (m²/g), more preferably 37 to 41 m²/g, and yet more preferably 39.9 m²/g. Other ranges are also possible.

At step 76, the method 70 includes heating the precursor material. In some embodiments, the precursor material is heated at a temperature of 350 to 450° C., preferably 380 to 420° C., or even more preferably about 400° C., to form the red mud-supported catalyst. In some embodiments, the heating can be performed by using heating appliances such as ovens, microwaves, autoclaves, hot plates, heating mantles and tapes, oil baths, salt baths, sand baths, air baths, hot-tube furnaces, and hot-air guns.

### EXAMPLES

The following examples demonstrate a method of converting furfural to a conversion product using a red mud-supported catalyst. The examples are provided solely for illustration and are not to be construed as limitations of the present disclosure, as many variations thereof are possible without departing from the spirit and scope of the present disclosure.

### Example 1: Materials

All chemicals, such as iridium (III) chloride hydrate (IrCl₃.xH₂O), rhodium (III) chloride anhydrous (RhCl₃), ruthenium chloride hydrate (RuCl₃.xH₂O), dichloromethane anhydrous (DCM), ethanol, and anhydrous toluene, were purchased from Sigma-Aldrich and used as received. Dry and deoxygenated solvents were also used.

### Example 2: Instruments

Lyra 3 was used for the field emission scanning electron microscope (FESEM) (manufactured by Tescan, Brno, Czech Republic). The FESEM samples were prepared on alumina stubs using double-sided conductive copper tape coated with gold. Energy-dispersive X-ray spectra (EDS) were collected using a Lyra 3 (TESCAN, Czech Republic) attachment to the FESEM for elemental identification and mapping. TEM images were obtained using a JEOL JEM2100F transmission electron microscope (manufactured by JEOL, Musashino Akishima, Tokyo, 196-0021 Japan. The TEM samples were prepared by dropping the samples from an ethanolic suspension onto a copper grid and allowing them to dry at room temperature. X-ray diffraction (XRD) data were collected using a Rigaku model Ultima-IV diffractometer and Cu-Kα radiation (1.5405 angstroms (A)) at 40 kilovolts (kV) and 25 milliamperes (mA) (manufactured by Rigaku, Japan) over a 20 range of 20° to 90°. The metal analysis of the RM sample was performed using an Agilent 7700 ICP-MS (Agilent Technologies, USA). An X-ray photoelectron spectroscopic (XPS) investigation used an X-ray monochromator with an Al-K micro-focusing microprobe (ESCALAB 250Xi XPS, Thermo Scientific, USA) to measure the surface composition and oxidation states of the samples. Base pressure was utilized to calibrate the binding energy scale. Using ASAP 2020 equipment from Micromeritics (Norcross, GA, USA), the surface area of the RM was measured using its nitrogen adsorption isotherm. The experiments were carried out in a liquid nitrogen bath at 77 kelvin (K). The pressure in the chamber was 2×10⁻⁹ torr. A Shimadzu 2010 Plus gas chromatograph and a mass spectrometer (GCMS, Japan) were used to identify catalytic products by matching the species to those in the Wiley Registry Mass Spectral Library, identifying them based on their molecular ion (M⁺) and detecting mass fragmentation. A BELCAT II analyzer (MicrotracBel, Osaka, Japan) was used to perform temperature-programmed reduction (TPR). For the H₂-TPR analysis, the catalyst was loaded with 50 milligrams (mg) and preheated for 30 minutes at 500° C. with an argon flow of 50 milliliters per minute (mL/min). The sample was heated to 40° C. After being exposed to a hydrogen and argon mixture (10% H₂ in Ar, 50 mL/min) over the catalyst, the sample was heated to 200° C. at a ramping rate of 10° C./min. Catalytic reactions were carried out in Teflon-lined autoclaves (HiTech, model M010SSG0010-E129A-00022-1D1101, USA) equipped with a pressure gauge and a mechanical stirrer.

### Example 3: Catalyst Preparation

The natural RM sample was dried at 110° C. for 1 hour (h) before being crushed and calcined for 5 h at three different temperatures (400° C., 500° C., and 600° C.) with a temperature gradient of 4° C./min, and the samples are denoted as RM-400, RM-500, and RM-600. Noble metals loading concentrations of 0.5%, 1%, 3%, and 5% (wt.%) on RM catalysts were prepared by physical mixing and grinding for 30 minutes, followed by heat treatment at various temperatures. M@RM-t denotes the developed catalysts with different metals and temperatures (M=weight percent of Rh, Ir, or Ru; RM=red mud; t=calcination temperature).

### Example 4: Procedure for Hydrogenation of Furfural Using the RM Catalyst

A high-pressure (100 bar) autoclave equipped with overhead magnetic stirring at a constant stirring speed of 250 rotations per minute (rpm) was used. Furfural (1 millimole (mmol), 88 microliters (μl)), 1% Rh@RM (10 mg) catalyst, and 10 mL dry ethanol were added to the reactor tube. The reactor was flushed three times with H2 before being pressurized to the desired level and heated to 120° C. for 24 hours at a continuous stirring speed of 250 rpm. The reactor was then cooled to room temperature and depressurized. The conversion and selectivity were measured by placing the sample in a vial, diluting the sample with DCM, and injecting the sample after dilution into Gas chromatography/Mass spectrometry (GCMS) using an HP-5 capillary column with 30meters (m) long, 0.32 millimeters (mm) diameter, and 0.25 micrometers (um) film thickness.

### Example 5: Characterization

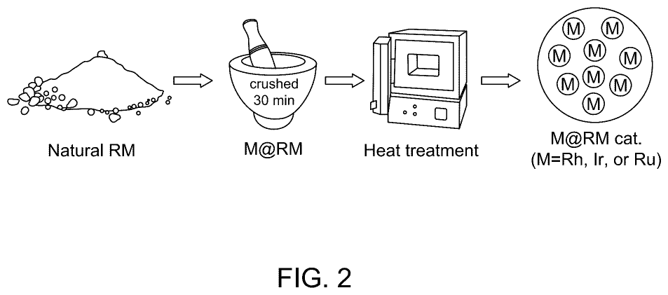

The steps involved in the catalyst preparation are shown in FIG. 2. The ground, red-colored RM granules were converted to powder and impregnated with the different weight percentages of the precious metals (M=Rh, Ir, and Ru) by the dry-mixing method. The mixture was calcined at 400° C. temperature with a gradient of 4° C./min with a holding time of 5 h, and samples were designated as 0.5% Rh@RM-400, 0.5% Ir@RM-400, and 0.5% Ru@RM-400. Samples calcined at 500 and 600°° C. are 0.5% Rh@RM-500 and 0.5% Rh@RM-600.

### Example 6: FESEM Analysis

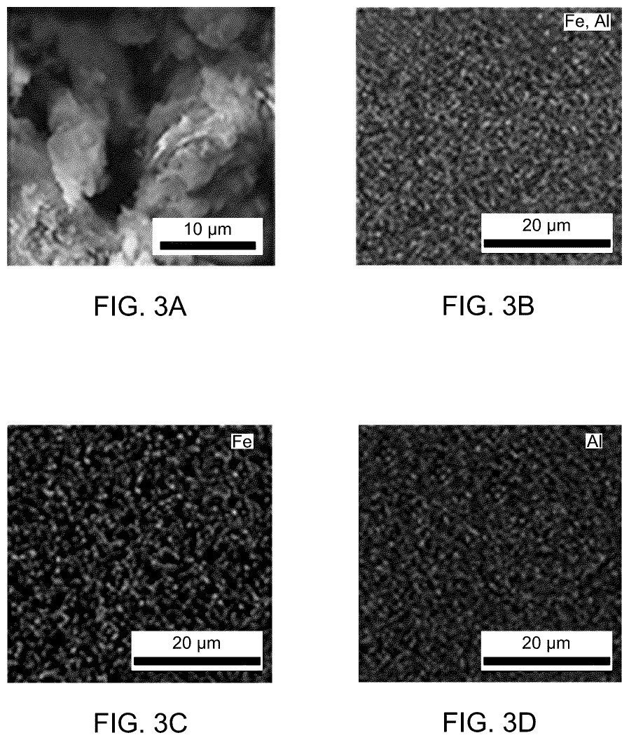

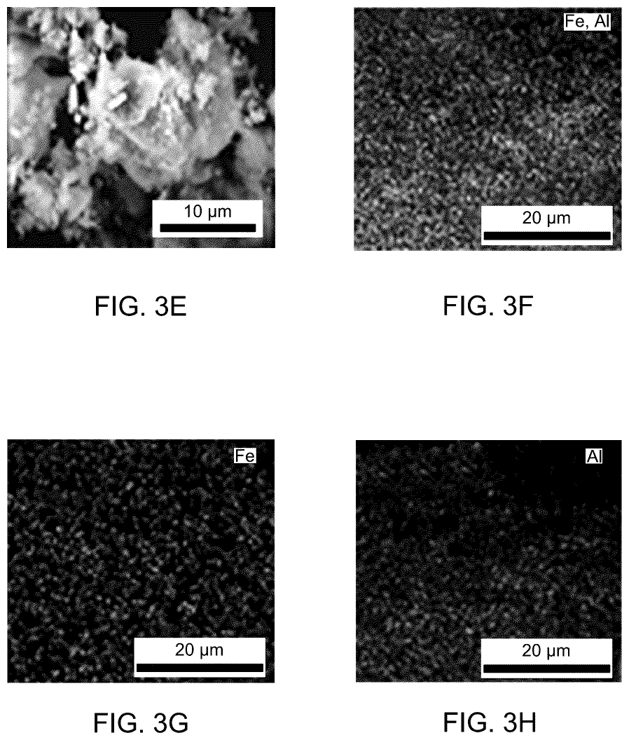

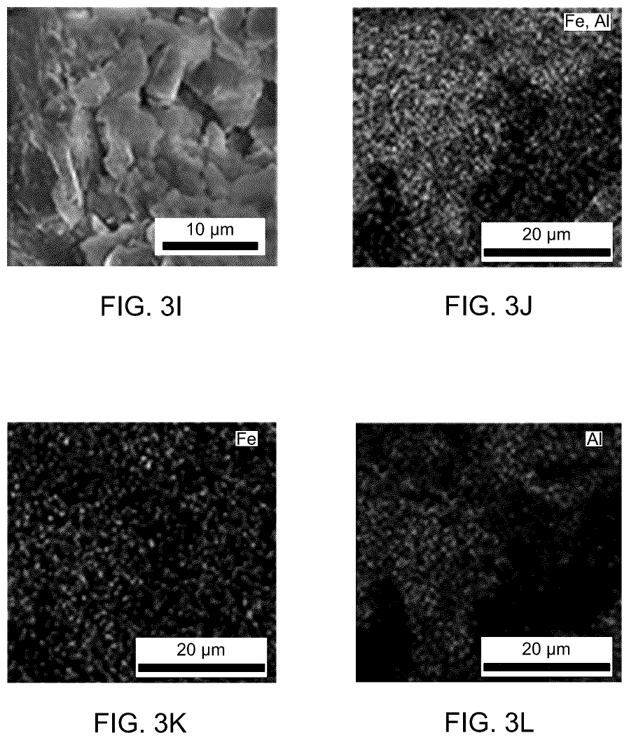

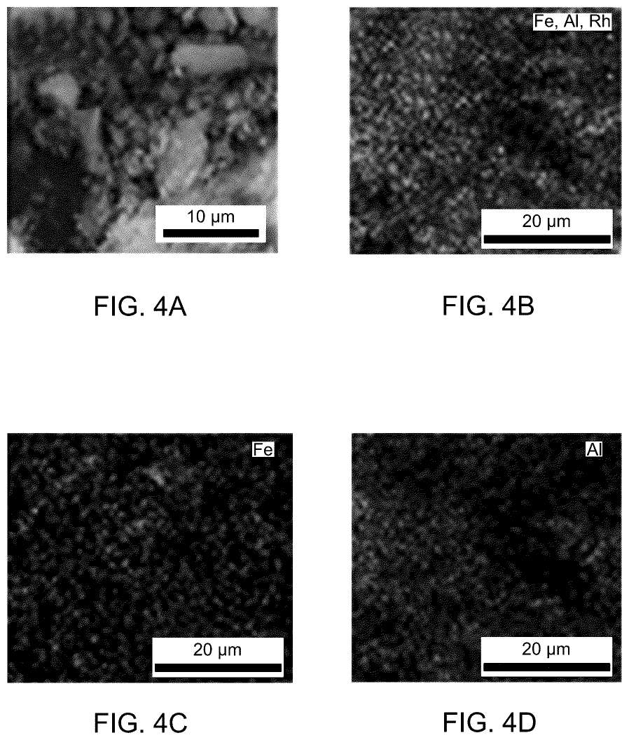

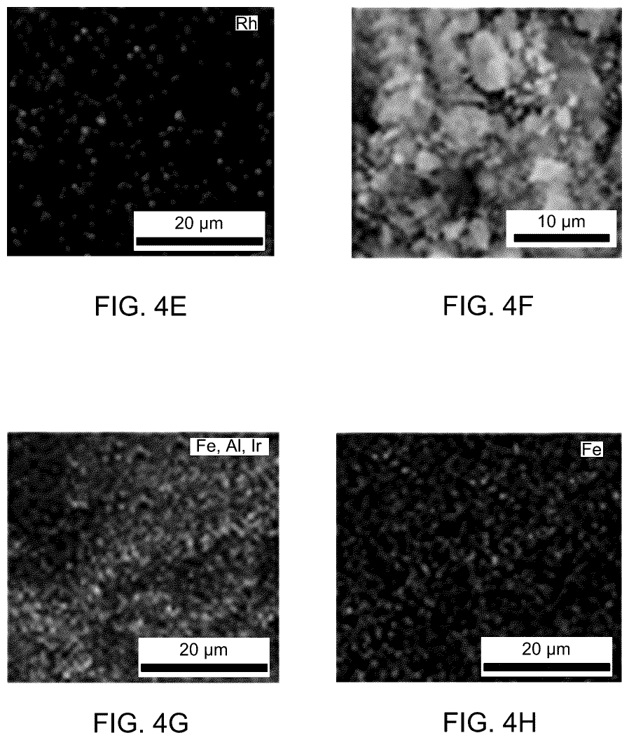

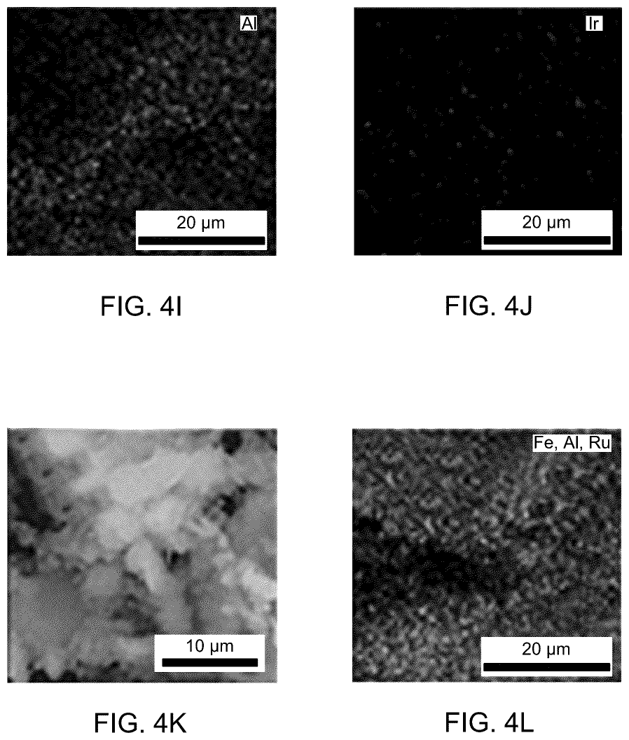

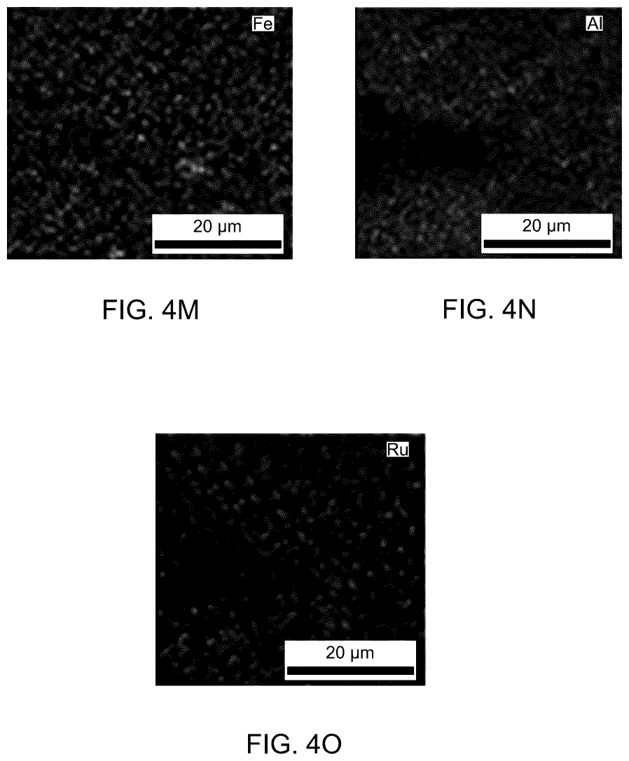

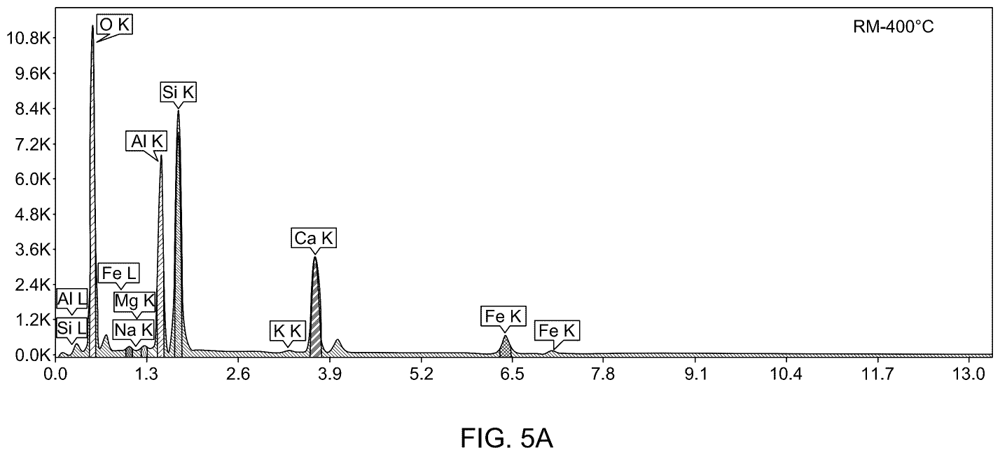

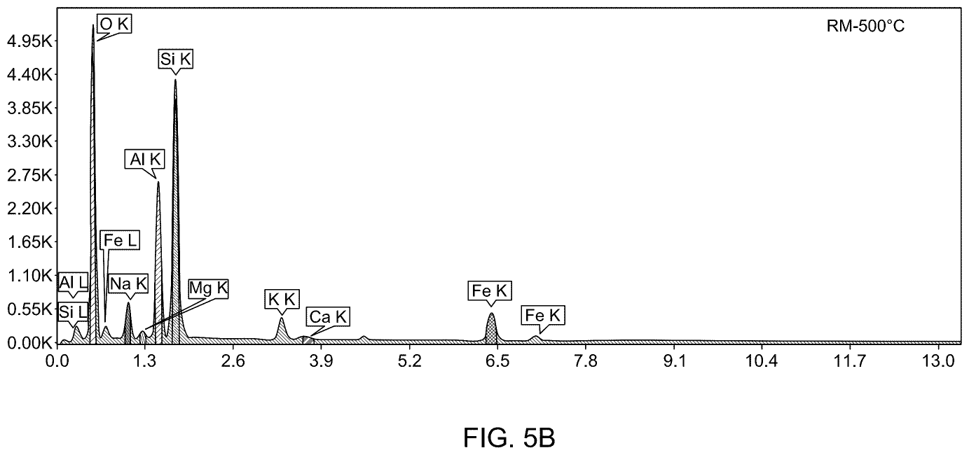

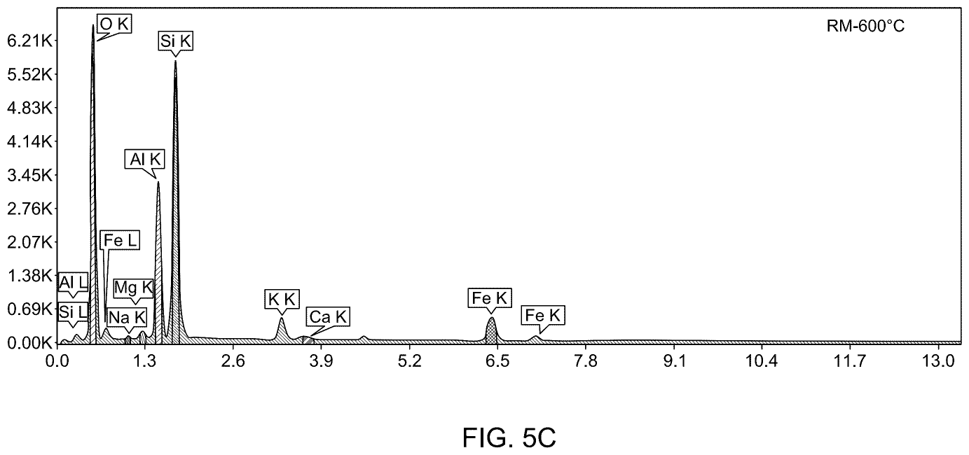

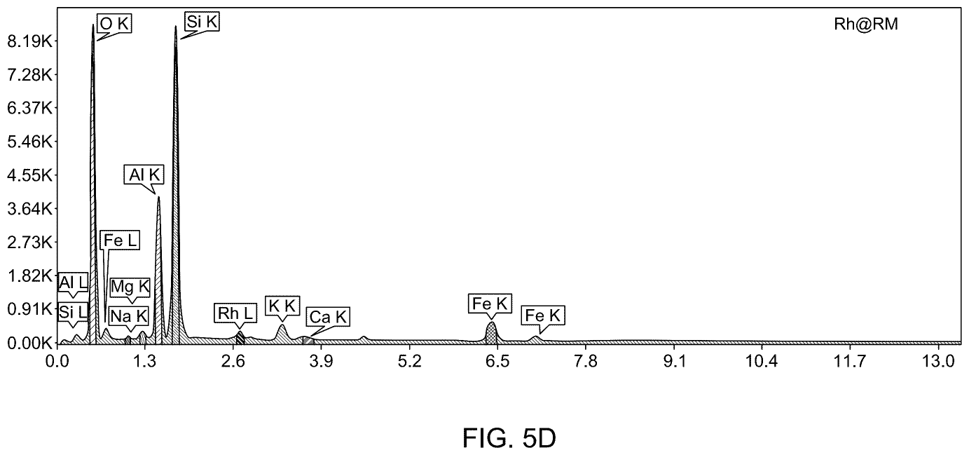

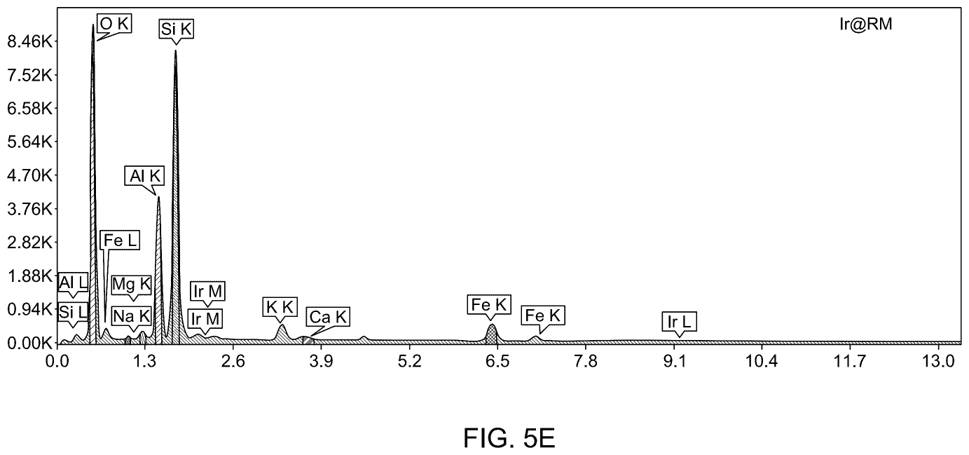

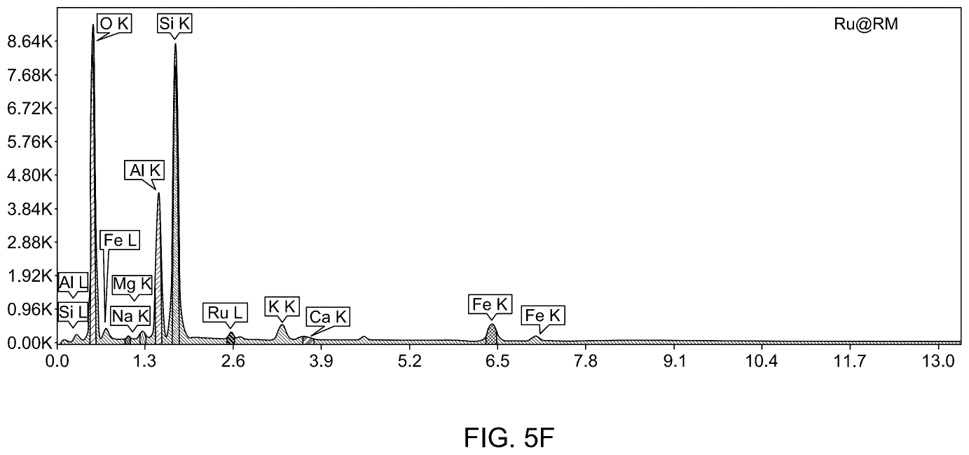

FIGS. 3A-3L and FIGS. 4A-40 depicts images obtained by FESEM and elemental mapping, highlighting the size, shape, and distribution. Their elemental identification is presented in FIGS. 5A-5F. The images demonstrate the morphology of the RM at three different temperatures. As the temperature increases from 400° C. (RM-400) (FIG. 3A) to 600° C. (RM-600) (FIG. 3E), smaller particles disappear and transform into bigger chunks (FIG. 3I). Primary elements, such as Fe (FIG. 3C, FIG. 3G, and FIG. 3K) and Al (FIG. 3D, FIG. 3H, and FIG. 3L), are mapped. The results show that both elements are distributed homogeneously throughout the samples for RM-400. A clear agglomeration of constituent elements is observed as the temperature increases from 400° C. to 600° C. Furthermore, when the FESEM images of RM-400, RM-500, and RM-600 with the impregnated noble metals such as Rh (FIG. 4A), Ir (FIG. 4F), and Ru (FIG. 4K) are compared, the crystal has a better-defined shape with clear surfaces, especially in case of

FIG. 4A. The elemental mapping reveals the homogeneous distribution of its major constituent elements Fe and Al in addition to the Rh in FIGS. 4B-4E. A similar trend in elemental distribution is noted with Ir (FIGS. 4H-4J), and Ru impregnated samples (FIGS. 4L-40).

### Example 7: TEM Analysis

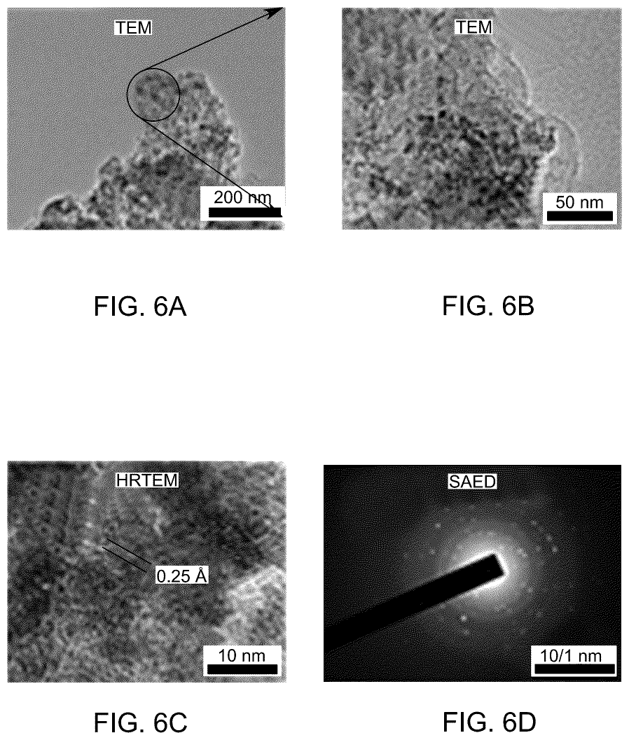

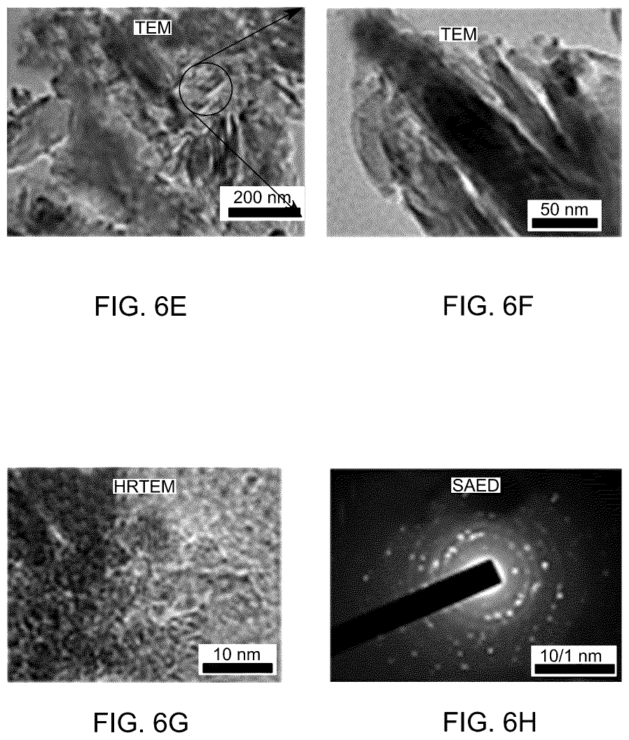

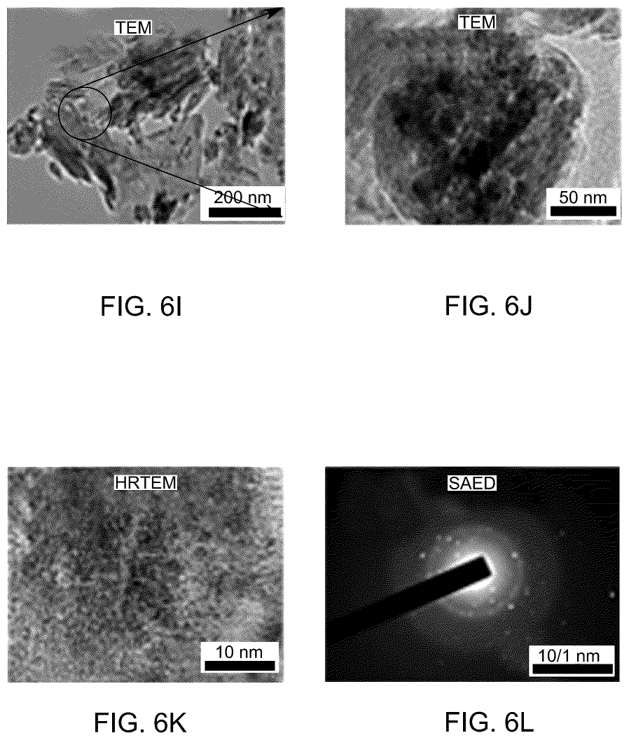

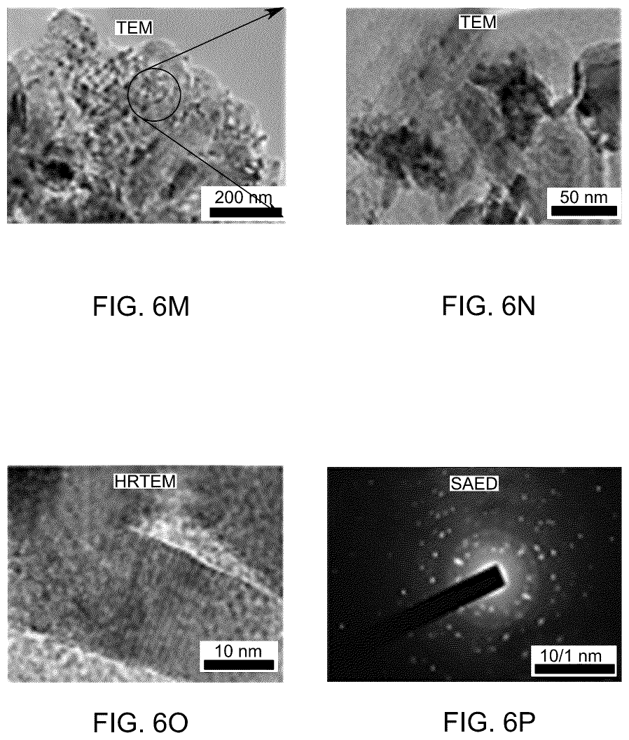

TEM analysis was performed for RM-400 and noble metal-impregnated samples, and the results are shown in FIG. 6A, FIG. 6E, FIG. 6I, and FIG. 6M). FIGS. 6A-6B and its corresponding HRTEM (FIG. 6C) lead to the identification of highly crystalline Fe₂O₃ with a fringe value of 0.25 nm in the RM-400 samples. The RM-400 catalyst impregnated with 0.5% Rh (FIG. 6E), 0.5% Ir (FIGS. 6I), and 0.5% Ru (FIG. 6M) were imaged, and the results exhibited a significant improvement in dispersion. Here, no substantial difference in morphology was observed when three different metals were loaded. However, the image for 0.5Rh@RM-400 reveals the needle-shaped fragmented particles consisting of various size ranges (FIG. 6E and FIG. 6F). The HRTEM shows that the sample loaded with 0.5 wt. % Rh is a mixture of crystalline and amorphous phase material (FIG. 6G).

In addition, HRTEM revealed ultrasmall dot-like Rh particles (<1 nm) (FIG. 6G) distributed throughout the area. The Bragg reflection is visible in selected area electron diffraction (SAED), which yields a lattice spacing d-value of 0.250 nm corresponding to the (311) plane of Fe₂O₃. The (012) plane of Al₂O₃ with a hexagonal crystal phase is responsible for a reflection of 0.335 nm.

### Example 8: XRD Analysis

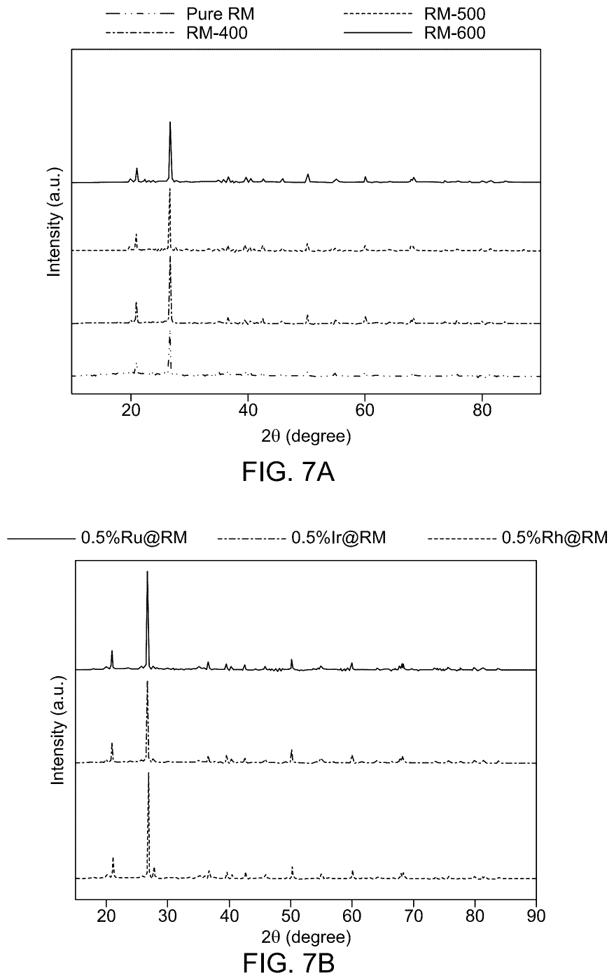

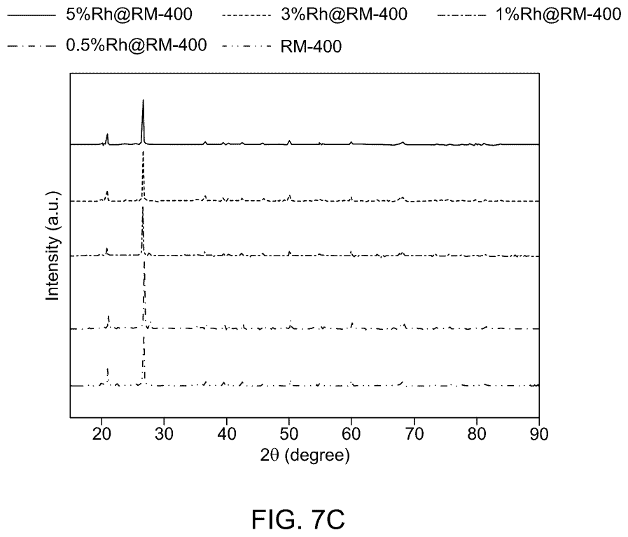

FIGS. 7A-7C show the XRD signatures of different RM catalysts prepared under varied conditions. FIG. 7A shows that it retains its crystalline nature throughout all the samples. However, one new increasingly prominent peak appeared at the diffraction range of 2θ=19° as the temperature was increased from 400° C. This signature pattern may correspond to a gibbsite ([Al(OH)₃]) formed by the stacking of the octahedral sheets of aluminum hydroxide (See: W. G. Shim, J. W. Nah, H.-Y. Jung, Y.-K. Park, S. C. Jung, S. C. Kim, Journal of industrial and engineering chemistry 2018, 60, 259-267, which is incorporated herein by reference in its entirety). The XRD also shows the crystalline phases of the main component of the RM catalysts. For instance, α-Fe₂O₃ is detected as hematite (Card No.: 01-073-2234), boehmite (AlO(OH)) with card no.: 01-072-0359), anatase titania (TiO₂, card No.: 01-071-1168), and gibbsite (Al(OH)₃ with card No.: 00-007-0324). No other foreign peak is detected in the XRD, indicating their composition is well-defined but dispersed. When a trace amount (0.5 wt. %) of noble metals, such as Rh, Ir, and Ru, is impregnated into the RM-400 sample (FIG. 7B), no new noble metal peak is observed for all three impregnated catalysts, showing homogeneous dispersion of noble metal nanoparticles on the RM-400 surface (FIG. 7B). Additionally, a low amount (0.5 wt. %) of Rh compared to the RM-400 can be the reason for not appearing in the XRD. A similar trend continued as the loading of the Rh increased to 5 wt. % in FIG. 7C.

### Example 9: FTIR Analysis

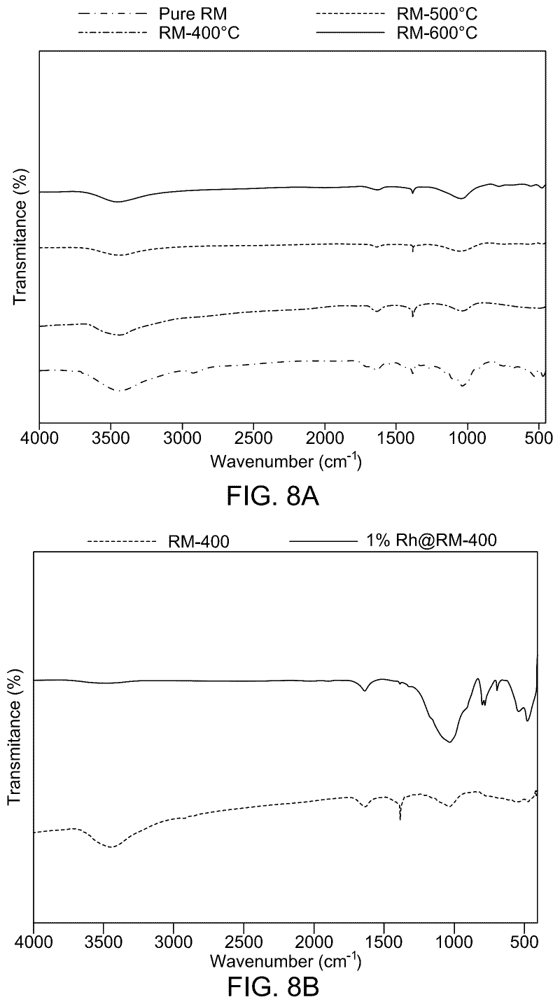

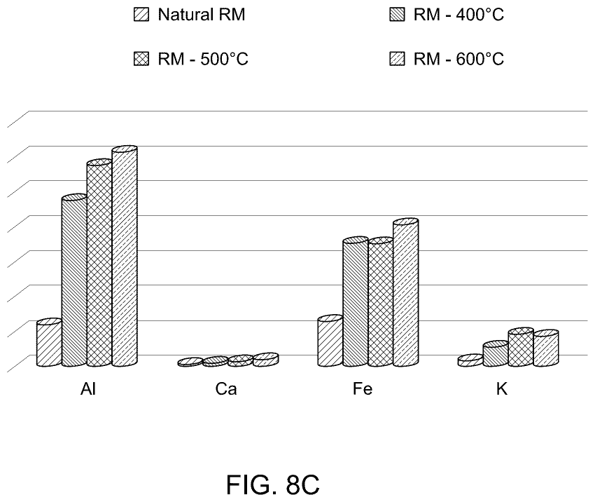

The Fourier transform infrared spectroscopy (FTIR) confirms the presence of functional groups in RM catalysts; the results are shown in FIGS. 8A-8C. The abundance of —OH functional groups from the water molecules in the uncalcined samples resulted in a highly pronounced peak at a 3400-centimeter inverse (cm⁻¹). However, it disappeared in the calcined samples, and the new stretching vibration for (Al)O—OH is observed at 3439 cm⁻¹. Furthermore, the stretching vibrations at 534 cm⁻¹ and 1629 cm⁻¹ identify Fe—O (See: B. Yuan, C. Bao, X. Qian, S. Jiang, P. Wen, W. Xing, L. Song, K. M. Liew, Y. Hu, Industrial & Engineering Chemistry Research 2014, 53, 1143-1149, which is incorporated herein by reference in its entirety). The presence of CaO is also detected at 1384 cm⁻¹.

### Example 10: TPR Analysis

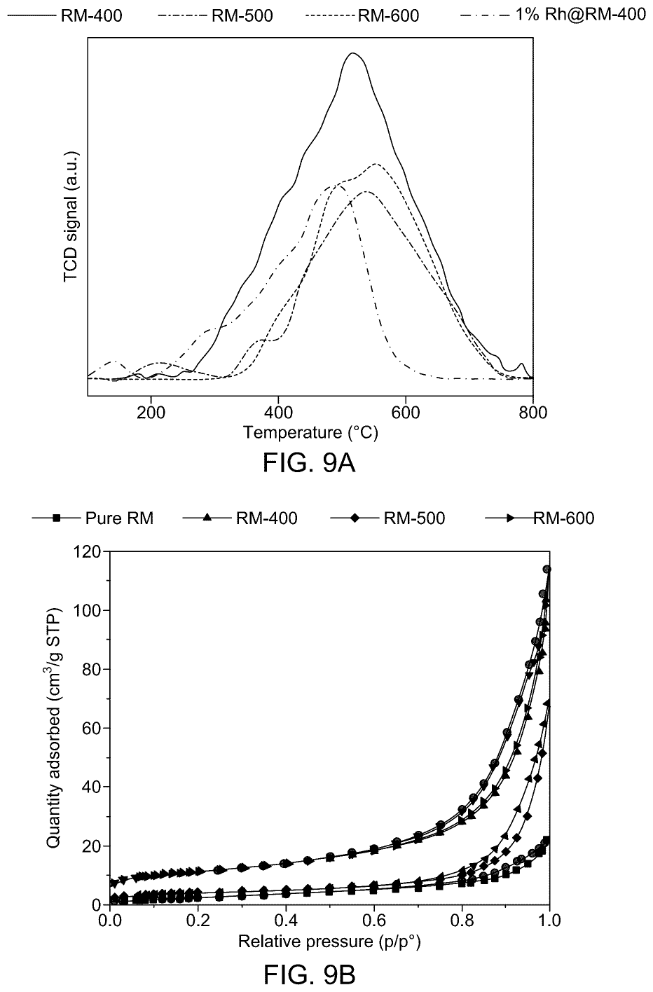

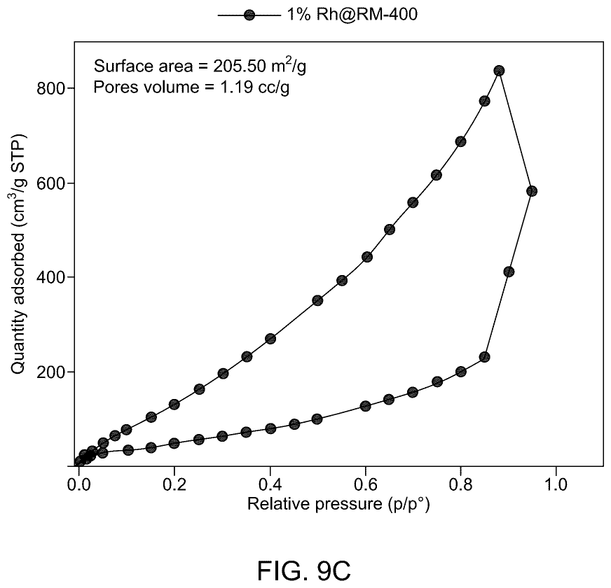

The TPR studies, as shown in FIG. 9A demonstrates the reduction profile of the constituent component in the RM. In the case of sample RM-400, a broad peak is observed in the range of 250° C. to 760° C., which is closely related to the continuous reduction of Fe₂O₃→Fe₃O₄→FeO→Fe⁰. This reduction phenomenon can be attributed to the gradual conversion of hematite to magnetite under the hydrogen environment as the temperature increases. At around 500° C., hematite was reduced entirely to magnetite. An increased amount of the magnetite was converted to zero-valent iron after reduction at 600° C. (See: A. Castille, C. Bessette, F. Thomas, M. Etemad, Catal Commun 2019, 121, 5-10, D. Spreitzer, J. Schenk, Steel Res Int 2019, 90, 1900108, which is incorporated herein by reference in its entirety). However, a similar trend is observed for the RM-600 sample, with a shorter temperature range (380-700° C.) and lower hydrogen consumption. The higher hydrogen consumption could be associated with the higher number of reducible species on the surface of the RM-400 catalyst. The surface area and porosity were measured, and the results are shown in FIG. 7C. Natural RM, RM-400, RM-500, and RM-600 had Brunauer-Emmett-Teller (BET)-specific surface areas of 10.9, 39.9, 14.3, and 39.4 m²/g, respectively, and the results are shown in FIG. 9B.

The surface area of RM-400 is greater than the surface area of pure uncalcined RM. This could be due to eliminating many volatile compounds from the surface of the RM at 400° C., drastically improving the surface area. Generally, the higher temperatures caused the sintering of the different species, leading to the reduction of both surface area and pore volume. However, the surface area (205.5 m²/g) resulting from 1% Rh@RM-400 catalyst was higher than RM-400 (FIG. 9C). It may be attributed to the higher number of tiny Rh nanoparticles (<1 nm) well-dispersed on the surface of the RM-400 surface. Hence, it leads to higher catalytic activity towards the hydrogenation reaction because of a higher number of exposed active sites of the Rh nanoparticles.

### Example 11: XPS Analysis

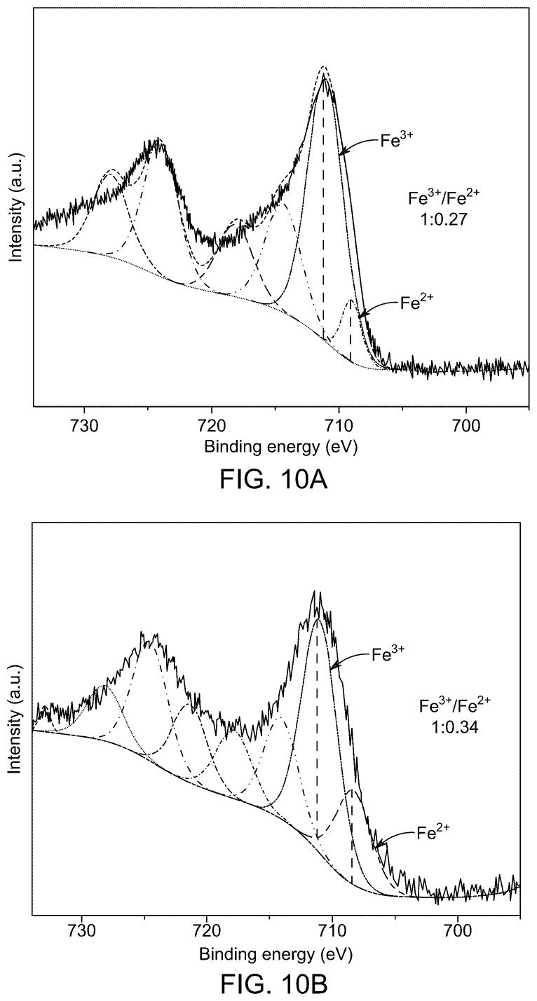

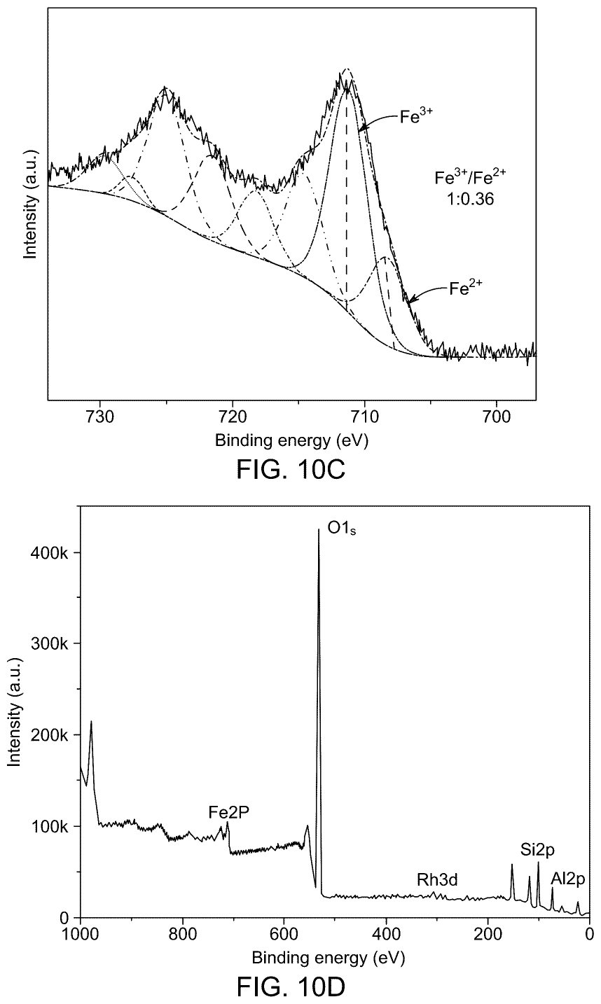

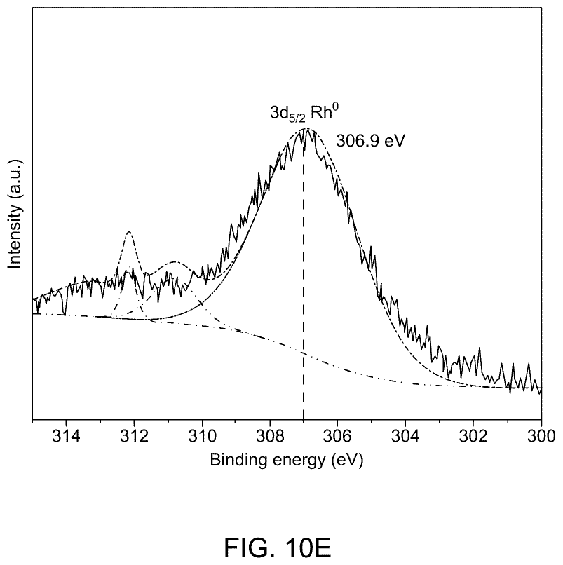

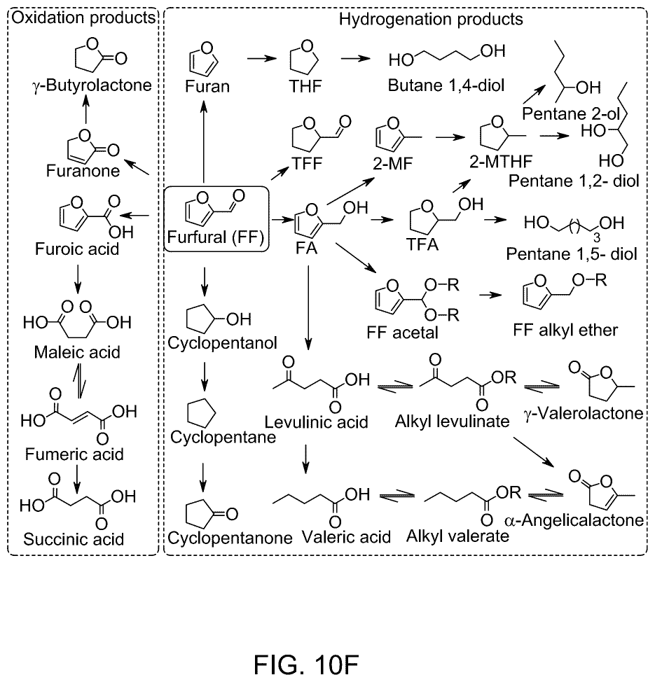

X-ray photoelectron spectroscopy (XPS) analysis was performed on the RM-400, RM-500, RM-600, and 1% Rh@RM-400 to examine the chemical composition and oxidation states of the iron in the composition, and the results are shown in FIGS. 10A-10E. The FIG. 10A shows that peaks at binding energies 710 electron volts (eV) and 724 eV correspond to the Fe 2p_{3/2} and Fe 2p_{1/2}(where Δ=14 eV). A weak shoulder satellite peak at 719 eV can be observed in the RM-400 sample, indicating the presence of Fe³⁺ species (FIG. 10A) (See: T. Yamashita, P. Hayes, Applied Surface Science 2008, 254, 2441-2449, which is incorporated herein by reference in its entirety).

For the samples calcined at 500 (RM-500) (FIG. 10B) and 600° C. (RM-600) (FIG. 10C), no shoulder satellite peaks were detected, which shows the decrease of Fe³⁺ species. The different

Fe species were identified by the deconvoluted Fe 2p spectra, indicating the peaks at binding energies 711.2 eV, showing the presence of Fe (III) species in the form of hematite(Fe₂O₃) and goethite (FeOOH) in the RM-400 catalyst. As the calcination temperature increased, the ratio of Fe³⁺/Fe²⁺ decreased, which may be caused by the conversion of Fe₂O₃ and FeOOH to Fe₃O₄. However, XPS confirmed the presence of an increased amount of magnetite species at lower binding energies, around 709.5 eV. The presence of Rh 3d was detected for the 1% Rh@RM-400 catalyst, as shown in the FIG. 10D. The oxidation states of the added Rh on the red mud are investigated (FIG. 10E). The binding energy of Rh 3d_{5/2} appeared at 306.9 eV and is associated with the metallic Rh⁰ on the red-mud support.

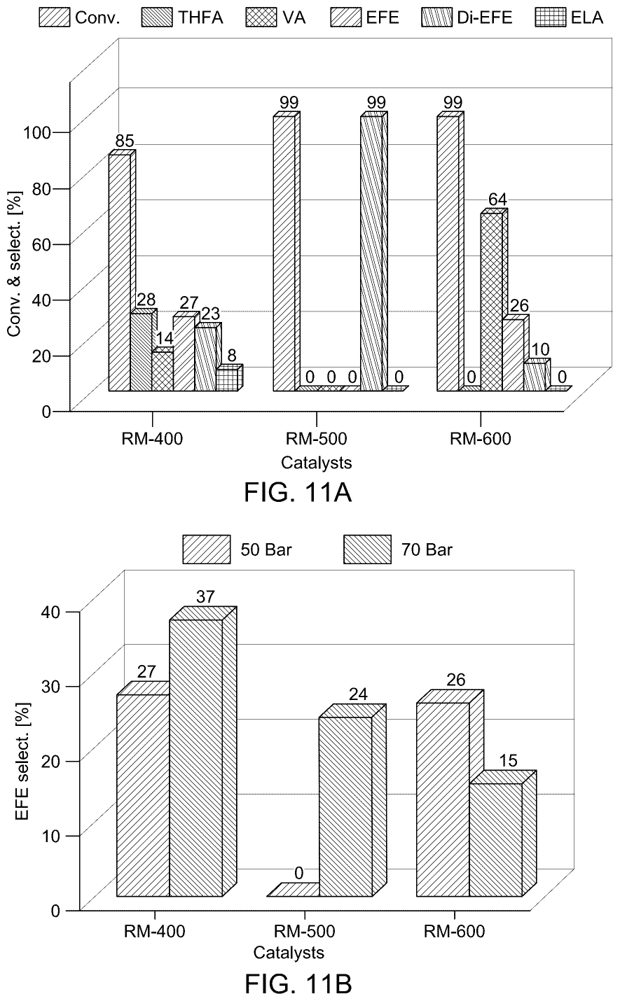

FIG. 10F shows the possible reaction routes for the furfural conversion to multiple products depending on the reaction condition. Under hydrogenating conditions, two functional groups available for the hydrogenation to produce their corresponding products, such as furfuryl alcohol (FA), tetrahydrofurfuryl alcohol (THFA), Diethyl-furfuryl ether (Di-EFE), (also known as acetal) and ethyl furfurylether (EFE). The reactivity of the catalysts RM, RM-400, RM-500, and RM-600 was evaluated by measuring the conversion and selectivity, and the results are shown in FIGS. 11A-11B. The catalyst requirement was established by running the reaction in the absence of the catalyst, and the conversion of the furfural did not occur. Then, the reaction condition was screened since the product selectivity is highly dependent on the parameters, such as temperature, pressure, and duration of the reaction.

### Example 12: The Effect of the Solvent on RM Catalyst Performance

In a liquid-phase reaction, the solvent often affects the catalytic activity. A comparison of toluene, DCM, and ethanol as solvents in terms of influence on catalytic performance in the hydrogenation of furfural was studied at 120° C. under 50 bar H₂ using the pure RM. The results showed no conversion in toluene or DCM (Table 1). At the same time, ethanol gave complete conversion (>99%) to 28% THFA, 14% valeric acid (VA), 27% EFE, and 23% Di-EFE, indicating a more promising route for furfural hydrogenation in a polar solvent than in moderate or nonpolar solvents.

TABLE 1
Effect of solvent on furfural hydrogenation^{a} using RM catalyst

| | | | | | Select. [%] | | | |
|---|---|---|---|---|---|---|---|---|
| | | Temp | H₂ | Conv. | | | | Di- |
| Catalyst | Solvent | [° C.] | [bar] | [%] | THFA | VA | EFE | EFE |
| Pure | DCM | 120 | 50 | 9 | nd | nd | nd | nd |
| RM | Toluene | | | <5 | nd | nd | nd | nd |
| | Ethanol | | | >99 | 28 | 14 | 27 | 23 |

^{a}10 mg pure RM catalyst under 50 bar hydrogen pressure at 120° C. for 24 h.
nd: Not determined;
THFA: Tetrahydrofurfural;
VA: Valeric acid;
EFE: Ethylfurfuryl ether;
Di-EFE: Diethylfururfyl ether

### Example 13: The Effect of Calcination Temperature on RM Catalytic Performance

A series of RM was prepared and calcined at different temperatures, and its reactivity and selectivity were evaluated and the results are shown in FIGS. 11A-11B. Since, calcination temperatures influence the electronic properties of the catalysts by a particular phase formation and its extent of crystal structure, surface area, and porosity. RM-400 catalyst was employed for the furfural hydrogenation in ethanol, and 85% of the furfural was converted to 27% EFE selectivity at 120° C. under 50 bar H₂ pressure (FIG. 11A). When the same reaction was conducted using RM-500 catalyst, >99% conversion of furfural ended with (>99%) diethyl furfuryl ether selectivity in 24 h. However, multiple product formation was noted when the RM was calcined at 600° C. (RM-600) with a reasonable amount of VA (64%). However, the EFE selectivity was recorded as 26% with RM-600 catalyst (FIG. 11A). The EFE selectivity was improved to 37% once the H₂ pressure was increased to 70 bar. This could be due to the higher dissolution of hydrogen at enhanced pressure, leading to improved EFE selectivity. Moreover, 24% EFE selectivity was noted with RM-500 catalyst at 70 bar.

However, the reverse trend in EFE selectivity was observed with RM-600 catalyst under the same reaction conditions. Additionally, a diverse product distribution was observed. For instance, RM-600 produces 52% diethyl furfurylether, 15% EFE, 18% valeric acid, and 11% ethyl levulinate (ELA). Although, valeric acid is a commercially attractive compound as it is produced in one step from bio-mass derived furfural through an environmentally benign route. Also, levulinic acid is an important building block for methyltetrahydrofuran (MTHF), which is a miscible oxygenate with improved vapor pressure properties (See: J. J. Bozell, L. Moens, D. C. Elliott, Y. Wang, G. G. Neuenscwander, S. W. Fitzpatrick, R. J. Bilski, J. L. Jarnefeld, Resources, Conservation and Recycling 2000, 28, 227-239, which is incorporated herein by reference in its entirety).

The higher reactivity of the RM-400 catalyst may be due to its higher surface area (BET 39.9 m²/g). The low calcination temperature regime of the RM tends to preserve its appropriate crystal phase in a multi-component system, such as SiO₂, Al₂O₃, CaO, Fe₂O₃, and Na₂O, and strikes a balance between phase formation and active sites, which leads to efficient diffusion of furfural and accessibility to active sites of the RM-400s. Additionally, the catalyst calcined at higher temperatures, such as RM-500 and RM-600, could lead to agglomeration or some degree of sintering, resulting in loss of active sites and compromising the catalytic performance. A higher content of Fe³⁺ species in RM-400, as evidenced by XPS, MAY BEresponsible for the high catalytic activity (See: R. M. Mironenko, V. P. Talsi, T. I. Gulyaeva, M. V Trenikhin, O. B. Belskaya, Reaction Kinetics, Mechanisms and Catalysis 2019, 126, 811-827; M. Farrag, Microporous and Mesoporous Materials 2018, 257, 110-117; C. Milone, C. Crisafulli, R. Ingoglia, L. Schipilliti, S. Galvagno, Catalysis today 2007, 122, 341-351, each of which is incorporated herein by reference in their entireties).

### Example 14: The Effect of the Reaction Temperature and Pressure on RM Catalytic Performance

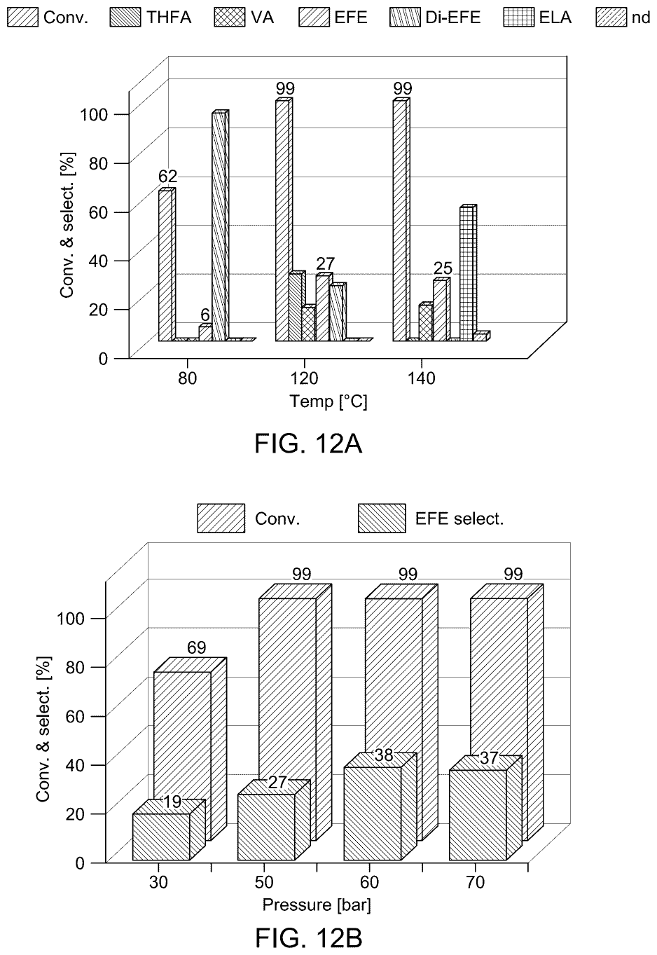

The effect of temperature on the hydrogenation reaction using RM-400 catalyst under 50 bar hydrogen pressure, and the results are summarized in FIG. 12A. Firstly, the reactivity of RM-400 catalyst at 80° C. was tested and produced 62% furfural hydrogenated product with 94% diethyl furfuryl ether (Di-EFE) selectivity in 24 h. As the temperature increased to 120° C., a complete conversion (>99%) of furfural occurred with 37% EFE and 14% VA selectivity, respectively. The product distribution was profoundly affected as the temperature increased further. For example, at 140° C., the EFE selectivity (25%) remained almost unaffected, but the ethyl levulinate selectivity (55%) increased. Then, the effect of hydrogen pressure on the catalytic furfural hydrogenation reaction was studied at 120° C. in ethanol as a solvent, and the results are shown in FIG. 12B. At 30 bar pressure, 69% furfural was hydrogenated with very low EFE selectivity (19%). An improvement (>99%) in hydrogenation reactivity was observed when the pressure increased to 50 bar with 27% EFE formation. This may be due to increased hydrogen solubility at higher pressure, leading to the effective interaction between furfural and the RM-400 catalyst. This enhanced hydrogen solubility also facilitates the higher diffusion of hydrogen into the catalyst and improves the overall kinetics of the reaction. Further increasing the pressure at 60 bar, 38% EFE, and 39% Di-EFE selectivity were recorded by GCMS. Unexpectedly, a different trend in product distribution was demonstrated when the reaction was conducted at 70 bar of hydrogen, where a reasonably higher amount (49%) of ethyl levulinate was formed with 37% of EFE. This higher pressure affects the reaction kinetics, leading to ethyl levulinate formation.

### Example 15: The Effect of Noble Metal Loadings on RM Catalytic Performance

RM as a catalyst, the furfural hydrogenation reaction was extended to maximize the selectivity towards the value-added chemicals, such as EFE, levulinic acid (LA) or ethyl levulinate from biomass-derived furfural. Precious metals, such as Rh, Ir, Pd, Pt, and Ru, are known to exhibit catalytic activity than any metals available in the periodic table. All these metals possess unique electronic structures and surface properties that enable efficient hydrogen adsorption and dissociation, resulting in higher catalytic activity and selectivity. Also, the different precious metals often preferred to be associated with certain functional groups. For instance, Pd metal, preferably hydrogenate —C=C— over the —C═O functional group when present in the same molecule. Therefore, numerous reports of precious metals anchored onto a range of solid supports, including graphene, porous carbon, metal oxides, and zeolites, have appeared in the literature and achieved excellent results in the catalytic hydrogenation reaction. However, the effect of precious metal on RM for furfural hydrogenation is rarely explored.

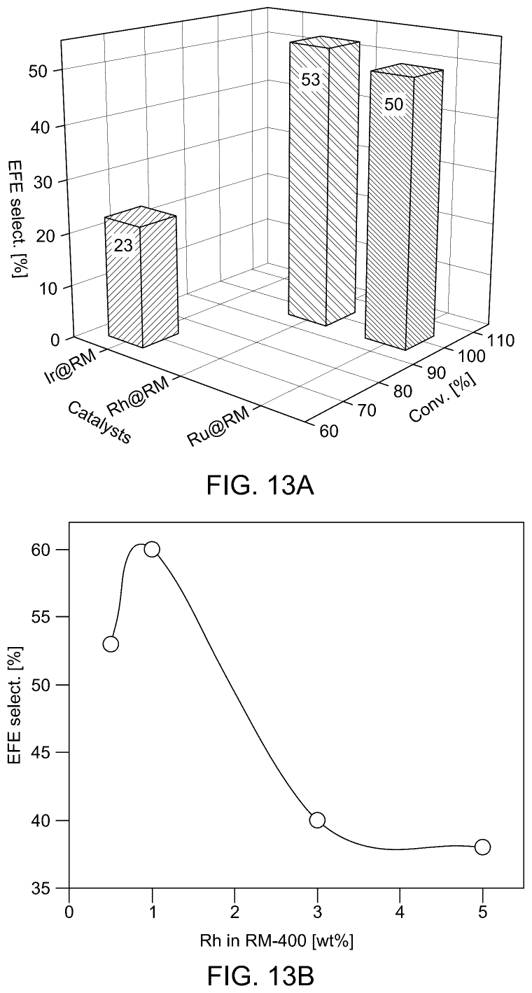

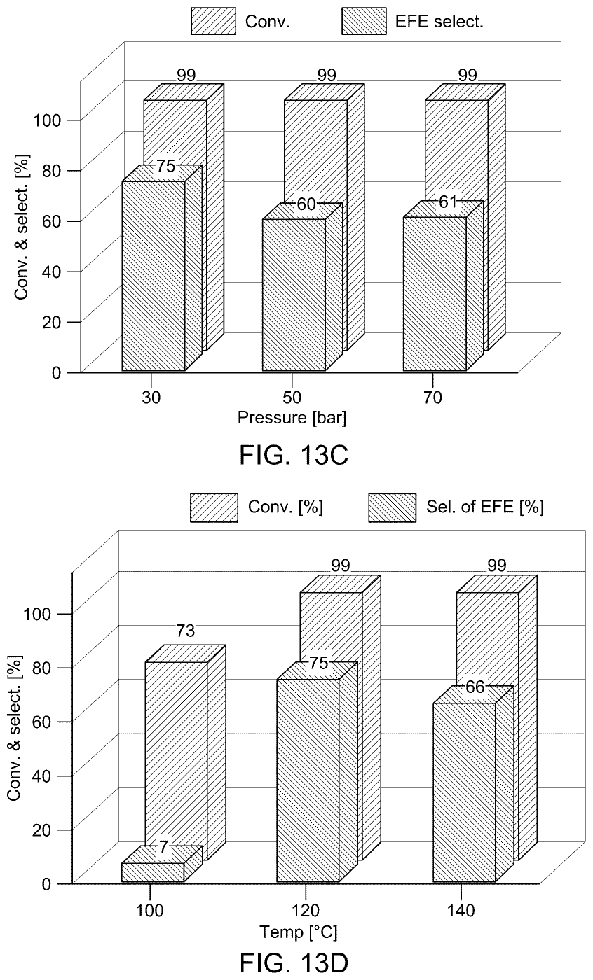

In the present disclosure, an amount, e.g., preferably about 0.5 wt. % of various precious metals, such as Rh, Ir, and Ru, were impregnated into the best-performing catalyst, RM-400, for the furfural hydrogenation reaction under the desired condition, and the results are summarized in FIGS. 13A-13D. Results showed that furfural, e.g., preferably about 1 mmol, was fully converted to the various hydrogenated products with 23% EFE selectivity using, e.g., preferably about 10 mg 0.5% Ir@RM-400 catalyst at 120° C. in ethanol in 24 h (FIG. 13A). An improvement in EFE selectivity (50%) was observed using 0.5% Ru@RM-400 catalyst as shown in FIG. 13A. When 0.5% Rh@RM-400 catalyst was employed for the hydrogenation, a further enhancement in EFE selectivity (53%) was detected by GCMS. Hence, it can be seen that 0.5% Rh@RM-400 effectively catalyzed to produce better EFE selectivity amongst the used precious metals for the furfural hydrogenation reaction at 120° C. in ethanol under 50 bar hydrogen in 24 h (FIG. 13A). In line with this effort, the amount of Rh content in the RM-400 was varied from 0.5 wt. % to 1, 3, and 5 wt. %, and the EFE selectivity was measured. In FIG. 13B, a quantitative conversion of furfural with 60% EFE selectivity was recorded using the 1wt. % Rh@RM-400. As the Rh content was increased to 3 wt. %, the EFE's selectivity diminished to 55%, and the decreasing trend in the EFE selectivity (45%) was continued with the increasing amount (5 wt. %) of Rh content in RM-400 under the same reaction condition. Hence, the optimum amount (i.e., 1 wt. %) of Rh nanoparticles may be finely dispersed on the surface of the RM-400 and tend to exhibit higher catalytic activity and, thereby, higher EFE selectivity due to increased accessibility and availability of active sites. Then, the 1 wt. % Rh@RM-400 catalyst was tested under different hydrogen pressures with the results shown in FIG. 13C.

The highest EFE selectivity (75%) was achieved under 30 bar of hydrogen at 120° C. using 1 wt. % Rh@RM-400 catalyst. Further increasing the hydrogen dissolution from 50 to 70 bar, the EFE selectivity decreased to 60 and 61%, respectively. The higher hydrogen pressure probably promotes random hydrogenation among two competitive functional groups present in furfural. Furthermore, with the increased availability of hydrogen in the reaction system under high pressure, the adsorption and desorption kinetics of hydrogen from the active metal surface are hindered, leading to changes in the availability of active sites. Also, the mass-transfer limitation cannot be ruled out under excessive pressure, which may disrupt EFE selectivity. The effect of temperature on furfural hydrogenation using 1 wt. % Rh@RM-400 was also tested, and the results are shown in FIG. 13D. The catalyst 1 wt. % Rh@RM-400 exhibited the best performance at 120° C. under 30 bar of hydrogen in ethanol with a duration of 24 h. By increasing the hydrogenation reaction temperature further to 140° C., the EFE selectivity decreased (66%). Thus, the bio-mass derived furfural can be quantitatively hydrogenated under 30 bar hydrogen pressure at 120° C. to produce 75% EFE as a petroleum blend using 1 wt. % Rh impregnated red mud (1 wt. % Rh@RM-400). FIGS. 14A-14T depict gas chromatography of hydrogenation of furfural at 120° C., under 20, 30, and 50 bar H₂ pressure in DCM and ethanol as solvents in 24 h using RM, 0.5% Ir@RM, 0.5% Rh@RM, 0.5% Ru@RM, 1% Rh@RM, 3% Rh@RM, 5% Rh@RM, as catalysts and identification of products by GCMS of the peak at different peaks.

### Example 16: The Reusability of 1 wt. % Rh@RM-400 in the Hydrogenation of Furfural

The stability of the 1 wt. % Rh@RM-400 catalyst was examined using reusability tests under the desired reaction condition. FIG. 15 displays the findings of the study. After the 1^{st} round of the hydrogenation reaction, the spent catalyst was collected, washed with ethanol, and then reused for successive consecutive cycles. The 1 wt. % Rh@RM-400 retains its reactivity in terms of conversion till, e.g., preferably about 6^{th} cycle and starts decreasing the furfural conversion slowly and 90% conversion was recorded after, e.g., preferably the 8^{th} cycle. However, the selectivity of EFE began to decline with an increase in Di-EFE and reached 37% of EFE after the 6^{th} cycle (FIG. 15). During the 8^{th} cycle, the selectivity of 1 wt. % Rh@RM-400 decreased to 4%. This drop-in activity can be attributed to the monotonous loss of the material during the handling of the reaction and the deactivation of some active sites after repeated use of the catalyst.

The red mud (RM) was incorporated in the production of ethyl furfuryl ether (EFE) as a petroleum blend. The utilization of RM as a catalyst emphasized as a beneficial alternative to conventional commercial catalysts. The present disclosure disclosed the effectiveness of an RM support catalyst in the selective hydrogenation of FF while also examining the effects of reaction conditions, reduction temperature, and metal loading. The RM-400 catalyst was found to be more selective than other catalysts, producing fewer by-products after the hydrogenation reaction. The behavior of hydrogen reducibility (H₂-TPR) was studied, and the results showed that the RM-400 catalyst has a large surface area and a large number of active sites on its surface, which may be caused by the increased amount of reducible species and hydrogen consumed. The results showed the role of noble metals (Ir, Rh, and Ru) supported on RM-400 in the selective production of EFE. In terms of EFE selectivity, the 0.5% Rh catalyst delivered higher (53%) than the counterpart 0.5% Ir (23%), and the 0.5% Ru (50%). The study also examined the variations in EFE selectivity using RM-400 with different Rh concentrations, specifically 0.5, 1, 3, and 5%. The results showed that 1% Rh@RM-400 produced the highest selectivity (75%) at 120° C. under 30 bar H₂. Reusability studies were conducted to evaluate the stability of the 1 wt. % Rh@RM-400 catalyst. The catalyst exhibited consistent catalytic activity over five cycles, maintaining a selectivity of over 60% EFE with complete FF conversion. The method of the present disclosure provides an approach for fabricating catalysts from the waste material, red mud, and selective production of biofuel, EFE, from the bio-mass derived furfural may pave the way for industrial application.

Numerous modifications and variations of the present disclosure are possible in light of the above teachings. It is therefore to be understood that within the scope of the appended claims, the invention may be practiced otherwise than as specifically described herein.

1. A method of converting furfural to a conversion product, comprising:
introducing furfural, an alcohol solvent and a red mud-supported catalyst to a reactor and mixing to form a mixture; and
introducing a hydrogen-containing gas into the reactor and contacting with the mixture thereby reacting the hydrogen of the hydrogen-containing gas and furfural in the presence of the red mud-supported catalyst to form the conversion product;
wherein the red mud-supported catalyst is at least one of a red mud-supported rhodium (Rh@RM) catalyst, a red mud-supported iridium (Ir@RM) catalyst, and a red mud-supported ruthenium (Ru@RM) catalyst.

2. The method of claim 1, wherein the alcohol solvent is at least one selected from the group consisting of methanol, ethanol, propanol, butanol, pentanol, hexanol, heptanol, octanol, nonanol, decanol, undecanol, and dodecanol.

3. The method of claim 2, wherein the alcohol solvent is ethanol.

4. The method of claim 1, wherein the hydrogen-containing gas further comprises an inert gas selected from the group consisting of nitrogen, argon, and helium.

5. The method of claim 1, having a furfural conversion of at least 60% based on an initial weight of the furfural present in the mixture.

6. The method of claim 1, wherein the reactor is a fixed-bed reactor in the form of a cylindrical reactor comprising:
a top portion;
a cylindrical body portion;
a bottom portion;
a housing having an open top and open bottom supportably maintained with the cylindrical body portion;
wherein the red mud-supported catalyst is supportably retained within the housing permitting fluid flow therethrough;
at least one propeller agitator is disposed in the bottom portion of the reactor;
wherein the bottom portion is cone shaped or pyramidal; and
wherein a plurality of recirculation tubes fluidly connects the bottom portion of the cylindrical reactor with the cylindrical body portion of the cylindrical reactor.

7. The method of claim 1, wherein the reacting is performed at a temperature of 80 to 160° C.

8. The method of claim 1, wherein the reacting is performed under a pressure ranging from 5 to 100 bar.

9. The method of claim 1, wherein the red mud-supported catalyst is a Rh@RM catalyst, and wherein the Rh@RM catalyst comprises about 0.5 to 5 wt. % of Rh based on a total weight of the Rh@RM catalyst.

10. The method of claim 1, wherein the red mud-supported catalyst is a Rh@RM catalyst, and wherein the Rh@RM catalyst comprises irregular-shaped particles and needle-shaped particles having an average diameter of 30 to 80 nanometers (nm).

11. The method of claim 1, wherein the red mud-supported catalyst is a Rh@RM catalyst, wherein Rh nanoparticles of the Rh@RM catalyst are uniformly distributed on surfaces of the Rh@RM catalyst, and wherein the Rh nanoparticles have an average particle size of less than 1 nm.

12. The method of claim 1, wherein the conversion product comprises furfuryl alcohol (FA), valeric acid (VA), tetrahydrofurfuryl alcohol (THFA), diethyl-furfuryl ether (Di-EFE), and ethyl furfurylether (EFE).

13. The method of claim 12, wherein the EFE is present in the conversion product in an amount of 30 to 80 wt. % based on a total weight of the conversion product.

14.: The method of claim 1, further comprising:
preparing the red mud-supported catalyst by:
calcining a red mud material at a temperature of 400 to 600° C. to form a calcined material;
grinding and mixing a metal salt and the calcined material to form a precursor material; and
heating the precursor material.

15. The method of claim 14, wherein the red mud material is a waste product from an aluminum extraction process.

16. The method of claim 14, wherein the red mud material comprises one or more crystalline phases selected from the group consisting of hematite, boehmite, anatase titania, and gibbsite, as determined by X-ray diffraction (XRD).

17. The method of claim 14, wherein the calcined material has a Brunauer-Emmett-Teller (BET) specific surface area of from 5 to 50 square meters per gram (m²/g).

18. The method of claim 14, wherein the metal salt is at least one selected from the group consisting of an iridium salt, a rhodium salt, and a ruthenium salt.

19. The method of claim 14, wherein the metal salt is present in the precursor material in an amount of 0.1 to 1 wt. % based on a total weight of the precursor material.

20. The method of claim 14, wherein the heating is performed at a temperature of 350 to 450° C.
